(19) United States
(12) Patent Application Publication
(10) Pub. No.: US 2011/0287479 A1
(43) Pub. Date: Nov. 24, 2011

(54) PROTEASE CATALYZED IN SITU END CAPPING OF OLIGOPEPTIDES IN AQUEOUS MEDIA

(75) Inventors: Richard A. Gross, Plainview, NY (US); Kodandaraman Viswanathan, Chennai (IN)

(73) Assignee: Polytechnic Institute of New York University, Brooklyn, NY (US)

(21) Appl. No.: 13/106,132

(22) Filed: May 12, 2011

Related U.S. Application Data
(60) Provisional application No. 61/334,311, filed on May 13, 2010.

(51) Int. Cl. C12P 21/06

(52) U.S. Cl. 435/68.1

(57) ABSTRACT

One-pot biotransformations give oligo(γ-L-Et-Glu) decorated with selected amine-functionalized end-groups at C-termini with a first process controlling the end group structure of peptides synthesized by protease catalyzed peptide synthesis, and a second process incorporating end-groups that can be used directly or after further modification as polymerizable entities. Papain, bromelain, α-chymotrypsin, Multifect P-3000 and Purafect prime 4000L are used as catalysts for oligomerization of γ-L-(Et)₂-Glu in the presence of mono functional amines. The series of amine nucleophiles (NH₂—R, acyl acceptors) mimic phenylalanine in that they possess aromatic rings linked to amine groups by one or more methylenes. Generally, addition of increased quantities of NH₂—R from 0 to 30, 50 and 70 mol % with respect to γ-L-(Et)₂-Glu results in decreased %-yield but increased mol % of NH₂—R end-capped oligo(γ-L-Et-Glu)-NH—R (determined by NMR). Irrespective of the protease used, 2-thiophene methyl amine (TPMA) gave the highest fraction of oligo(γ-L-Et-Glu)-NH—R chains. l-phenylalanine and L-histidine did not produce end-capped oligo(γ-L-Et-Glu) and, inn contrast, L-phenylalanine analogs benzylamine (BzA) and L-phenylalaminol (F—OH), both of which lack the α-carboxyl group, gave substantial quantities of oligo(γ-L-Et-Glu)-F—OH, or -BzA chains. The promiscuity of proteases can be exploited to create a diverse family of desired end-functionalized oligopeptides. MALDI-TOF spectra recorded of oligo(γ-L-Et-Glu) with amine nucleophiles showed molecular ions that affirmed the formation of corresponding NH₂—R functionalized oligo(γ-L-Et-Glu).

### STATEMENT OF RELATED APPLICATIONS

This application is based on and claims the benefit of U.S. Provisional Patent Application No. 61/334,311 having a filing date of 13 May 2010, which is incorporated herein in its entirety by this reference.

### BACKGROUND OF THE INVENTION

1. Technical Field

This invention relates generally to an enzymatic process for preparing ‘C’ and ‘N’ terminal modified oligopeptides and relates more specifically to peptide based macromonomer synthesis in a one pot reaction.

2. Prior Art

Peptides are functionally rich molecules which can be designed to exhibit a wide range of biological activities and physico-mechanical properties and much more. Peptides with functional terminal groups have been used as monomers to develop important new protein mimetic materials. They have also been appended to surfaces to create a range of novel properties. Conventional synthesis of peptides involves solid phase or solution phase synthesis using multiple steps of protection and deprotection. A drawback of these methods is they involve multiple steps (protection, deprotection), use of harsh chemicals and racemization of the substrates often occurs during coupling steps of amino acid substrates.

Synthesis of novel peptide-containing materials is an active area of investigation showing great promise. Collagen, keratin, elastins and silk are structural proteins that exhibit properties commensurate to their function in nature. The attractive properties of these proteins results from their well-defined three-dimensional structure which is governed by primary amino acid sequences. Attempts have been made to mimic silk-inspired beta sheet elements in block copolymers.

Borrowing from the repetitive motifs in these structures, work has been performed to prepare macromers with end-group polymerizable entities and a peptide consisting of a repeated sequence within the protein. For example, a methaacrylate monomer bearing an AGAG sequence prepared by solid phase synthesis was copolymerized with methaacrylic acid by atom transfer radical polymerizations¹. Synthetic peptide-based vaccines have been prepared by copolymerization of acrylated peptides with multiple B and T-cell epitopes with acrylamide². Model oligolysine and oligo(glutamic acid) functionalized polyphenylene dendrimers were prepared to study DNA complexation and as building blocks for the electrostatic layer by layer self assembly of novel supramolecular architectures³. Kim et al. (2003) used peptide segments in acrylate copolymers in an attempt to develop biodegradable tissue scaffolds for the generation of an artificial extracellular matrix material (ECM).⁴ Proteolitically cleavable peptide sequences were acrylated and used as degradable crosslinkers in hydrogels formed by copolymerizations with N-isopropylacrylamide and acrylic acid. These peptide containing materials are plagued by difficulties in preparing peptides by tedious step-by-step solid and solution chemical peptide synthetic methods. If such macromers could be prepared directly from amino acid alkyl esters by protease catalysis, the preparation of peptide-containing hybrid materials could be considered for a much wider range of new material needs.

One approach known in the art is to use enzyme catalysis to modify peptide terminal groups. For example, a β-peptidyl aminopeptidase was used to catalyze the conjugation of β-amino acids to the free N-termini of other β-amino acids and α-tripeptide. Furthermore, these workers showed the peptidase catalyzed bond formation between β- and α-amino acids to the α-peptide H-Val-Ala-Leu-OH.⁵ Lack of peptide stability in biological milieus is a major hurdle to their use as therapeutics. As a solution, peptide N-terminal units have been modified via acetylation or methylation^{6,7}. All the above represent multistep processes as well as peptide synthesis by conventional chemical methods that are tedious and costly.

Therefore, it can be seen that there is a need for novel enzymatic processes for preparing ‘C’ and ‘N’ terminal modified oligopeptides and to peptide based macromonomer synthesis in a one pot reaction. It is to this need, and other needs, that the present invention is directed.

### BRIEF SUMMARY OF THE INVENTION

This invention describes an innovative one-pot route to end-functionalized oligopeptides using protease catalysis. The method of synthesis circumvents the tedious protection-deprotection steps required during conventional chemical peptide synthetic methods while providing access to a wide range of end-functionalized peptides structures. By preparing such macromers directly from amino acid alkyl esters by protease catalysis, the preparation of peptide-containing hybrid materials can be considered for a much wider range of new material needs.

This invention also describes a unique approach by which in-situ end-capping of peptides is accomplished during their synthesis by protease catalysis in a one-pot oligomerization reaction. The methodology described in this invention enables preparation of a wide range of end-capped peptides that vary in physico-chemical and biological properties. Examples are given by which the polymerizable end-group is varied. This allows polymerization by different methods such as free radical, various living polymerization methods and metathesis ring-opening polymerization.

Variation in monomer structure facilitates an ability to use a broad range of commoners that allows further diversification to peptide material structure. Also, living polymerization techniques enable control of structure, such as the ability to make block copolymer with defined segment length. Examples of desirable properties that can be obtained by proper design of the peptide, polymer composition, repeat unit sequence and polymer topology (e.g. multiarm, graft) is to create novel peptide containing materials that are: pH sensitive, have high drug binding capacities, biocompatibility, and antimicrobial properties.

Functionality at end groups also allows peptide to be appended to surfaces thereby tailoring their physical and biological properties. Peptides of this invention bearing specific terminal groups that endow them the ability to be polymerized, attached to surfaces, improve their efficacy as drugs or ingredients, or that themselves are biologically active. Examples of desirable biological properties attainable from this invention include but are not limited to antimicrobial, polymeric carriers of anticancer drugs, bind specific metals, provide beneficial attributes to skin (e.g. stimulate collagen growth). They are prepared by a unique one-pot methods by protease catalysis from amino acid alkyl esters and various molecules that only react such that they reside on either the C- or N-terminus of peptides.

Given that thiophene monomers are polymerizable and give conducting materials, routes to high purity 2-thiophene methylamine oligoglutamic acid can be used to prepare a wide range of oligopetptide functionalized conducting materials for medical applications.⁸ Furthermore the heterocyclic side chains themselves possess biological activity. For example, thiophene-2-carboxylic acid, an analog of 2-thiophene methyl amine used in this invention, is an effective inhibitor of bone resorption in tissue culture.⁹ Thiophene-2-carboxylic acid is known for its hypocalcemic effects, which inhibits calcium bone resorption.¹⁰ By using the methods of this invention furfural derivatives were appended to the end of a peptide. Furfuryl derivatives of phenoxyphenoxyalakanoic acids are known to have herbicidal properties (U.S. Pat. No. 4,404,818) and furfurylamide derivatives show fungicidal activities. Aminomethyl heterocyclic compounds are also used as pesticides and fungicides (U.S. Pat. No. 4,851,405) and can have metal chelation property as reported by the carboxy terminal peptides (International Patent Publication No. WO 01/52898). Furthermore, end-functionalized peptides prepared by the method described in this invention can be used for applications in nutrition and flavor chemistry.^{11,12}

The inherent advantage of enzymatic peptide synthesis has led to its evolution as an alternative to chemical coupling methods.¹¹ The thiol-protease papain is reported to be the most efficient catalyst for aqueous phase synthesis of homo-oligomers of hydrophobic amino acids like leucine, methionine, phenylalanine, and tyrosine.^{12,13} The equilibrium of such reactions is tilted in favor of synthesis by the precipitation of hydrophobic oligomers. In examples given in this invention, protease concentrations were normalized based on a common activity Unit.¹⁴⁻¹⁶ Casein assay was adopted to define the hydrolytic activity of different proteases used in this invention.¹⁷ An alternative assay method to quantify protease activity can be substituted for the casein assay and such methods are well known by persons of ordinary skill in the art. This overcomes the problem of using protease catalysts whose activity is not quantified in a way that would allow others skilled in the art to repeat their work.

There are a wide range of molecules that are known to one skilled in the art that would provide bioactivity, be polymerizable, chelate metals or provide some other valuable property when used in this invention to prepare peptides with end-functionalization during one-pot protease catalyzed oligopeptide synthesis from amino acid alkyl ester monomers. In Scheme 1 are shown structures and corresponding abbreviations of primary amine nucleophiles (NH₂—R) that function as acyl acceptors during one-pot concurrent oligopeptide synthesis and end-capping reactions. Scheme 1 also describes N-acryloyl-ethyl phenylalanine (AcF) as an example of an N-functionalized amine that served as an acyl donor to end-cap chains at their N-terminus during one-pot concurrent oligopeptide synthesis and end-capping reactions.

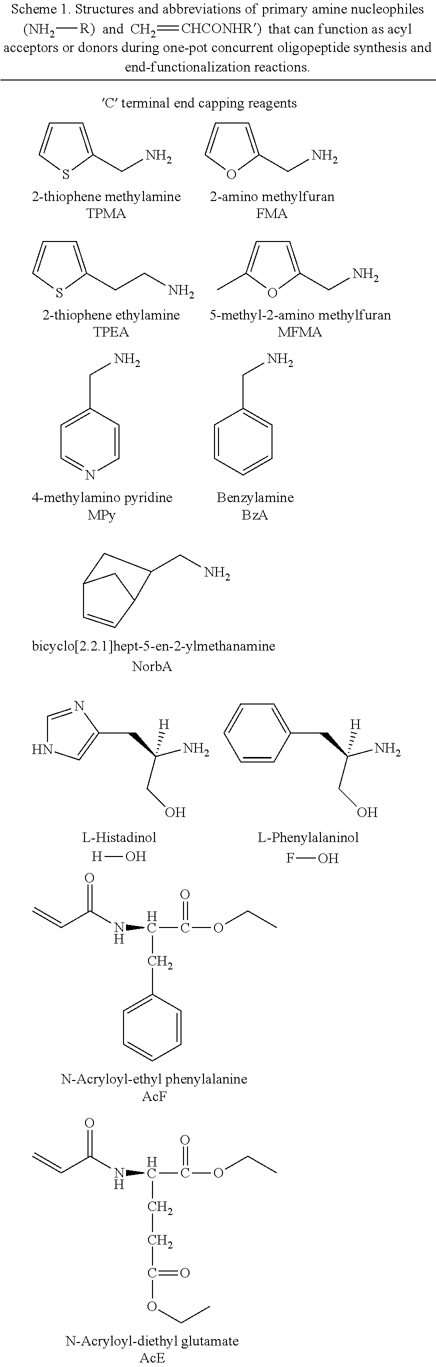

These and other objects, features, and advantages of the present invention will become more apparent to those of ordinary skill in the art when the following detailed description of the preferred embodiments is read in conjunction with the appended figures

### BRIEF DESCRIPTION OF THE SCHEMES, TABLES, AND FIGURES

Scheme 1. Structures and abbreviations of primary amine nucleophiles (NH₂—R) and CH₂═CHCONHR′) that can function as acyl acceptors or donors during one-pot concurrent oligopeptide synthesis and end-fuctionalization reactions.

Scheme 2. Proposed model of concurrent peptide synthesis and in-situ end-capping of the oligopeptides.

Table 1. %-Yield and %-oligo(γ-L-Et-Glu) modified at the ‘C’-terminus with R—NH₂ end-capping nucleophiles as a function of the γ-L-Et₂-Glu to R—NH₂ ratio, enzyme origin, and R—NH₂ structure.

Table 2. %-Yield and %-oligo(γ-L-Et-Glu) modified at the ‘C’-terminus with R—NH₂ end-capping nucleophiles as a function of the γ-L-Et₂-Glu to R—NH₂ ratio, enzyme origin, and R—NH₂ structure.

Table 3. %-Yield and %-oligo(γ-L-Et-Glu) modified at the ‘N’-terminus with R—COOEt end-capping acyl donor as a function of the γ-L-Et₂-Glu to R—COOEt ratio, enzyme origin, and R—COOEt structure.

FIG. 1. Hydrolytic activity of proteases determined by the casein hydrolysis assay (Sigma Aldrich technical bulletin product code PC0100).

FIG. 2. Protein content determined by the BCA assay (Pierce BCA assay kit protocol).

FIG. 3. ¹H-NMR (300 MHz, DMSO-d₆) spectra of oligopeptides synthesized with total monomer concentration of 0.5 M in 0.9 M phosphate buffer at 40° C. for 3 h: A) oligo(γ-L-Et-Glu) synthesized using Multifect P-3000 as catalyst; B) 2-thiophenemethylamine end-capped oligo(γ-L-Et-Glu) using papain as catalyst and feed ratio of 7:3 γ-L-Et₂-Glu-to-TPMA; C) 2-thiophenemethylamine end-capped oligo(γ-L-Et-Glu) using bromelain as catalyst and feed ratio of 7:3 γ-L-Et₂-Glu-to-TPMA; D) 2-thiophenemethylamine end-capped oligo(γ-L-Et-Glu) using Multifect P-3000 as catalyst and feed ratio of 7:3 γ-L-Et₂-Glu-to-2-thiophenemethylamine.

FIG. 4. MALDI-TOF spectra of products consisting of oligo(γ-L-Et-Glu)[Eₙ] and oligo(γ-L-Et-Glu)[Eₙ]-NH-TPMA, where ‘n’ represents the number of repeat units, prepared using the following proteases and γ-L-Et₂-Glu:TPMA molar feed ratios: A) Multifect P-3000, no TPMA; B) bromelain, 7:3; C) Multifect P-3000, 7:3, [MI] molecular ion peak, [DE] desterified peak, the m/z values are ±1 da of the expected m/z values.

FIG. 5. Evaluation of proteases for conversion of γ-L-(Et)₂-Glu to oligo(γ-L-Et-Glu) as a function of medium pH. Error bars represent the deviation from the mean of duplicate experiments

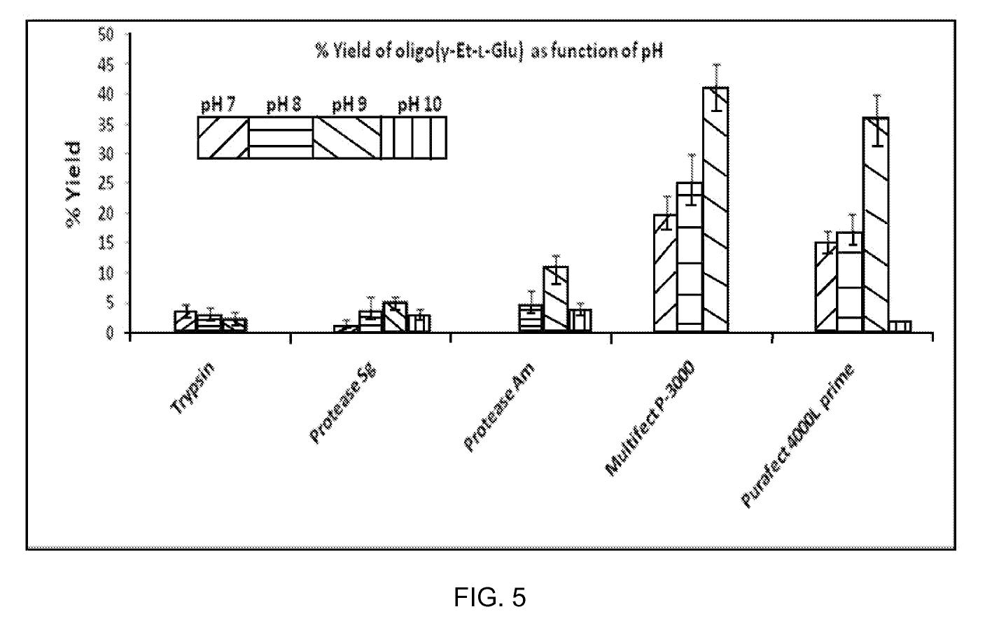

### DETAILED DESCRIPTION OF PREFERRED EMBODIMENTS

This invention is an innovative one-pot biotransformation for preparation of oligo(γ-L-Et-Glu) decorated at the C-terminus with amine-functionalized end-groups. Papain, bromelain, α-chymotrypsin, Multifect P-3000 and Purafect prime 4000L were used as catalysts for oligomerization of γ-L-Et₂-Glu in the presence of mono functional amines. Protease concentrations in reactions were normalized based on their activity for casein hydrolysis. The relative order of protease activities for oligo(γ-L-Et-Glu) synthesis is as follows: papain˜bromelain>α-chymotrypsin>Multifect P-3000>Purafect prime 4000L at pH optima values of for the proteases at 8, 8, 9, 9 and 9 respectively. Irrespective of the protease studied, addition of increased quantities of amine nucleophile relative to Et₂-Glu results in decreased %-yield but increased mol % of amine end-capped chains oligo(γ-L-Et-Glu)-NH—R. These trends were explained by that increasing the ratio of NH₂—R: γ-L-Et₂-Glu groups: i) can cause an increase in the quantity of chains formed and, correspondingly, a reduced oligo(γ-L-Et-Glu)-NH—R DP_{avg}, resulting in product that precipitates to a lesser extent; and ii) results in a reduced concentration of oligomerizable substrate in reactions. It also may be that NH₂—R functions as a competitive inhibitor of protease catalysts.

Scheme 2 illustrates a proposed model for the concurrent synthesis of end-capped oligo oligo(γ-L-Et-Glu) and displays competing reactions, which when properly manipulated lead to efficient end-capping of propagating oligopeptide chains. Several factors influencing the reaction highlighted within Scheme 2 include the promiscuity of protease used, concentration of the competing nucleophile, substrate concentration and pH of the solution. A highly efficient model is proposed wherein the acyl complex of the enzyme and substrate of choice reacts to an acyl acceptor (H₂O, NH₂—R or NH₂-γ-L-Et₂-Glu). Reaction between NH₂—R and γ-L-Et₂-Glu enzyme complex results in the formation of γ-L-Et-Glu-NH—R that subsequently propagates to form oligomeric product. Alternatively, oligomerization of γ-L-Et₂-Glu to oligo(γ-L-Et-Glu)ₙ followed by reaction with NH₂R to form oligo(γ-L-Et-Glu)ₙ-NH—R can lead to the desired end-functionalized. The latter can precipitate or continue to propagate adding x γ-L-Et₂-Glu units prior to precipitation (forming oligo(γ-L-Et-Glu)ₙ₊ₓ-NH—R). In case of H₂O acting as the acyl acceptor, the reaction leads to formation of α-C-terminal desterified product.

Regardless of the protease used, TPMA was the most efficient NH₂—R compound for reacting with α-ethyl ester moieties of γ-L-Et₂-Glu monomer or propagating oligo(γ-L-Et-Glu) chains thereby forming C-terminal functionalized oligopeptides. For all NH₂—R compounds used herein, data was obtained on how their efficiency for end-group functionalization differed as a function of the protease used. For example, with Et₂-Glu to TPMA of 7:3, the mol % of oligo(γ-L-Et-Glu)-TPMA formed with papain, bromelain, α-chymotrypsin, Multifect P-3000 and Purafect prime 4000L is 25, 50, 75, >95 and >95, respectively.

The extent of protease promiscuity as a function of NH₂—R structure was addressed. This provided insights into the range of amine nucleophiles that may be incorporated at oligo(γ-L-Et-Glu) C-termini. For example, TPMA and TPEA differ structurally by just one methylene unit between thiophene and amine groups. However, TPEA was a relatively poor amine nucleophile for oligo(γ-L-Et-Glu) end-capping. Indeed, only papain, with γ-L-Et₂-Glu to TPEA of 7:3 and 5:5, gave 20 and 25 mol % of oligo(γ-L-Et-Glu)-TPEA chains. Hence, for this series of amine nucleophiles and proteases, there appears to be a strong preference for using α-instead of β-amino acids.

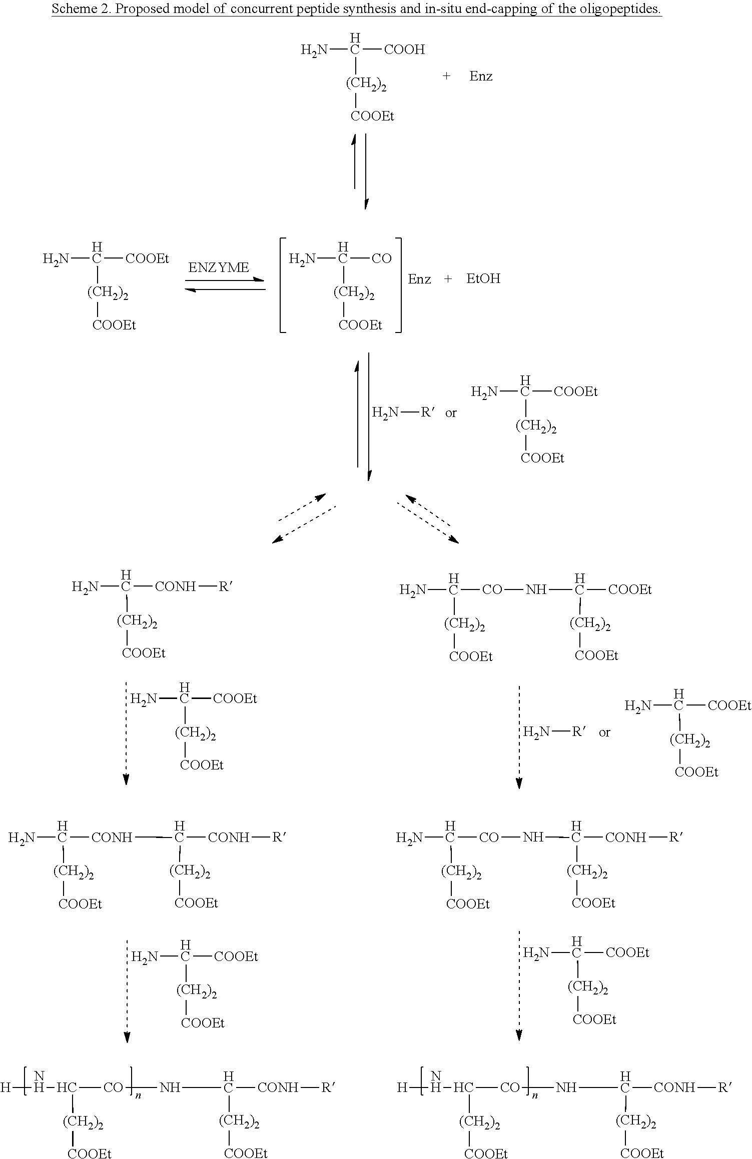

Using FMA in place of TPMA as the amine nucleophile probed the subtle change of replacing a thiophene with a furan heterocylic ring structure. While total product yields were similar with both these nucleophiles, TPMA was more efficient than FMA for C-terminal end-capping reactions. The affect of incorporating a methyl substituent at FMA furan ring 5-positions (e.g. using MFMA) was also investigated. For all 5 proteases, total yields were lower when using MFMA instead of FMA as amine nucleophile. However, in general, the efficiency of MFMA and FMA for C-terminal modification based on precipitated product was similar. In fact, for papain and γ-L-Et₂-Glu to R—NH₂ 5:5, incorporation of MFMA was higher than FMA (44 vs 20%).

Attempts to use either L-histidine or L-phenylalanine as amine nucleophiles were unsuccessful. To explore what structural characteristic of these compounds might result in this outcome, two L-phenylalanine analogs were tested where the α-carboxyl group was replaced by a hydrogen atom (BzA) and a CH₂—OH moiety (F—OH), respectively. At molar ratio of γ-L-Et₂-Glu-to-BzA of 3-to-7, the mol % of oligo(γ-L-Et-Glu)-BzA with papain and bromelain was 55 and 62%, respectively. At 7:3 L-Et₂-Glu to F—OH, catalysis by papain, α-chymotrypsin and Multifect P-3000 formed oligopeptides where oligo(γ-L-Et-Glu)-F—OH constituted 20, 30 and 45 mol % of total product. Thus, based on this limited set of substrates and enzymes, it appears that the presence of an α-carboxyl group on the amine nucleophile was deleterious to its function for amide formation with either γ-L-Et₂-Glu or propagating oligo(γ-L-Et-Glu) chains.

Typically, it was found that, in the absence of an amine nucleophile, protease catalyzed synthesis of oligo(γ-L-Et-Glu) occurred where MALDI-TOF analysis showed the formation of a substantial fraction of chains hydrolyzed at one of their ethyl ester pendant or end-group sites. If the site of ester hydrolysis is the C-terminus, then complete modification of the C-terminus by an amine nucleophile would give products with all other esters intact. Indeed, for oligo(γ-L-Et-Glu)-NH-TPMA synthesized by Multifect P-3000 catalysis, MALDI-TOF signals lacked low intensity peaks corresponding to one de-esterified ester group for oligo(γ-L-Et-Glu)-NH-TPMA. Therefore, it is believed that de-esterification observed primarily occurs at the α-carboxyl moiety at the C-terminus of oligo(γ-L-Et-Glu).

The cumulative results reported herein provide a unique general method that is simple and scalable by which oligopeptides can be prepared from protease catalysis from one or more amino acid alkyl esters in one-pot reactions with control of end-group structure. Furthermore, insights gained into protease promiscuity will allow the design of amine nucleophiles with desired functionality and high reactivity for incorporation at oligo(γ-L-Et-Glu) C-termini.

### EXPERIMENTAL

### Materials:

L-diethyl glutamic acid hydrochloride [L-(Et)₂-Glu.HCl] was purchased from Tokyo Kasei Co. Ltd. L-phenylalaminol and L-histidinol were purchased from Alfa Aesar. Bicyclo[2.2.1]-5-heptene-2-carbonitrile was purchased from Frinton laboratories.

Crude papain (cysteine protease EC 3.4.22.2; source, Carcica papaya; 30,000 USP units/mg of solid; molecular weight 21 K) was purchased from CalBiochem Co. Ltd. Water insoluble materials in the as-received papain were removed by dissolving 300 mg/mL crude papain powder in deionized water, centrifugation at 5000 rpm for 30 min, collecting the clear supernatant and discarding the insoluble precipitate. The clear supernatant was lyophilized overnight to obtain fully water-soluble papain as a beige powder that was used for all methods herein.

Bromelain (cysteine protease; EC 3.4.22.4; source pineapple stem; 3.4.22.32, protein content ≧35% protein by biuret, 1.7 units/mg protein), α-chymotrypsin type II (serine protease; EC 3.4.21.1; from bovine pancreas, 83.9 units/mg, 96 units/mg protein), Protease SG (serine protease), Trypsin (serine protease; EC 3.4.21.4; source bovine pancreas; ≧10,000 BAEE units/mg protein), Protease Subtilisin Carlsberg type VIII (serine protease, EC 3.4.21.14; source Bacillus licheniformis; 7-15 units/mg solid by casein assay), Protease Sg (serine protease; source, Streptomyces griseus; 4 units/mg solid by casein assay), Proteinase type XXVII (EC 3.5.1.14; source Aspergillus melleus; 3 units/mg of solid by casein assay) were purchased from Sigma Aldrich.

Multifect P-3000 (serine protease; IUB 3.4.21.62; from genetically modified strain of Bacillus subtilis; 2,750 GSU (Genencor subtilisin units/g), Purafect prime 4000L (serine protease; IUB 3.4.21.62; from Bacillus amyloliquefaciens; 4000 PPU (purafect prime units)/g), and Alkaline protease (serine protease) were kind gifts from Genencor International.

2-Thiophene methyl amine, 2-thiophene ethyl amine, 2-furfurylamine, 5-methyl-2-furfurylamine, benzylamine, 2-methylamino pyridine sodium phosphate diabasic, sodium acetate, casein, deuterated dimethyl sulfoxide (DMSO-d₆), Folin & Ciocalteu's reagent, trichloroacetic acid, and α-cyano hydroxycinnamic acid (CCA, MALDI-TOF matrix) were all purchased from Aldrich.

Deionized water (DI, 18.2 MΩ·cm purity) was obtained from a RIOS 16/MILLQ Synthesis Millipore water purification system.

All chemicals were purchased in the highest available purity and were used as received except when otherwise specified.

### Methods:

Determination of hydrolytic activity (Casein Assay). The following procedure follows that described in a technical bulletin for a protease colorimetric detection kit (product code PC0100 [Sigma Aldrich]). In summary, a dilute solution of the respective enzyme (25 μL) was transferred to a 1 mL microfuge tube. Casein solution (130 μL, 0.65%) was then added to the microcentrifuge tube and the solution was incubated at 37° C. for 10 min. Then, a solution of trichloroacetic acid (TCA, 130 μL, 110 mM) was added and the combined solution was further incubated for 20 min. Subsequently, the solution was centrifuged and 250 μL of supernatant was assayed by addition into a Na₂CO₃ solution (625 μL, 500 mM), with Folin & Ciocalteu's phenol reagent (125 μL). The absorption was measured at 660 nm. Activity was calculated as follows:

$$Units \text{/} mL enzyme = \frac{( μmol Tyrosine equivalents released ) ( A )}{( B ) ( C ) ( D )}$$

where:
A=total volume (mL) of assay
B=time of assay (min) as per the unit definition
C=volume of enzyme used (mL)
D=total volume (mL) used in colorometric determination.

Tyrosine equivalents in μmol released is determined by using the equation constructed from the standard graph for L-tyrosine (absorption vs concentration).

General procedure for protease catalyzed oligo(γ-L-Et-Glu) synthesis. The method for oligo(γ-Et-L-Glu) synthesis was adapted from a literature procedure.²² In summary, L-(Et)₂-Glu.HCl (600 mg, 2.5 mmol), protease (16 units/mL), and 5 mL of 0.9M sodium phosphate buffer solution set at a predetermined pH (commensurate with the optimum pH for oligo(γ-L-Et-Glu) synthesis for the protease used) was transferred to a 50 mL borosilicate glass tube, fitted with a TEFLON® cap and placed in a parallel reactor Carousel 12 (Radleys discovery technologies). Reactions were performed with gentle magnetic stirring at 40° C. for 3 h. The reaction mixture was cooled to room temperature, the resulting precipitated product was centrifuged and the supernatant was discarded. The precipitate was then washed first with deionized water (2×5 mL), then with an HCl solution (pH 2, 2×5 mL) and the remaining solid was lyophilized. Control experiments performed with substrates without addition of enzyme did not yield precipitate.

Synthesis and Characterization of C-terminus modified oligo(γ-L-Et-Glu). The method used is identical to that for synthesis of oligo(γ-Et-L-Glu) except for the following modifications. Although the stoichiometry of L-(Et)₂-Glu.HCl and end-capping substrate was varied, the total concentration of monomer and end-capping substrate remained at 2.5 mmol. Due to the high basicity/acidity of end-capping nucleophiles (NH₂—R), sodium phosphate buffer solution (5 mL, 0.9M) was added as above and solutions were titrated back to the desired pH using 10M NaOH or 10M HCl. Subsequently, 16 units/mL of the desired enzyme was added to catalyze oligopeptide synthesis. Precipitate formed was washed with 2×5 mL of de-ionized water then with an HCl solution (pH 2, 2×5 mL), separation of precipitate after each washing step was by centrifugation (5000 rpm), supernatants were discarded and the precipitate was freeze dried to obtain the product. The %-yield of amine end-capped chains of oligo(γ-L-Et-Glu) [oligo(γ-L-Et-Glu)-NH—R] was calculated gravimetrically from the precipitated product obtained.

¹H NMR chemical shifts (δ in ppm) in DMSO-d₆ of oligo(γ-L-Et-Glu): 1.16 (t, —CH₃), 1.84 (d, —CH₂), 2.30 (m, —CH₂), 3.78 (bs, —CH), 4.03 (m, —CH₂), 4.25 (bs, —CH), 7.8-8.6 corresponding to (—CONH) region of oligo(γ-Et-L-Glu).

¹H NMR chemical shifts (δ in ppm) in DMSO-d₆ of end-functional groups linked to oligo(γ-L-Et-Glu: 2-amino methylfuran (FMA): 6.2 ([(d, —CH], furan ring 3-position), 6.38 ([d, —CH], furan ring 4-position), 7.5 ([d, —CH], furan ring 5-position); 2-thiophene ethylamine (TPEA): 2.91 ([t, 2H, —CH₂] of TPEA), 6.9 ([bd, 2H, —CH], thiophene ring 1,3-positions), 7.3 ([bs, —CH], thiophene ring 4-position); 5-methyl-2-amino methylfuran (MFMA): 2.2 ([bs, —CH₃], substituted at furan ring 5-position), 5.9 ([bd, —CH], furan ring 4-position), 6.08 ([bd, —CH], furan ring 3-position); 4-methylamino pyridine (MPy): 4.07 ([m, —CH₂], substituted at pyridine ring 4-position and [—CH₂] region of the oligo[γ-L-Et-Glu]ester group), 7.22 ([m, 2H, —CH], pyridine ring 3.5 position), 7.8-8.6 ([m, 2H, —CH], pyridine ring 1.6 positions and [—CONN] region of oligo[γ-Et-L-Glu]); benzylamine (BzA): 4.1 (m, —CH₂, substituted at benzene ring 1-position and —CH₂ region of oligo[γ-Et-L-Glu]ester group, 7.28 (m, 5H, [—CH], benzene ring positions 2-6);

### Instrumental Methods:

Nuclear Magnetic Resonance (NMR) Proton (¹H) NMR spectra were recorded on a Bruker DPX 300 spectrometer at 300 MHz. Products (10 mg/mL) were dissolved in deuterated dimethyl sulfoxide (DMSO-d₆) and a total of 128 scans were collected and analyzed by MestRec-C software. Proton chemical shifts were referenced to tetramethylsilane (TMS) at 0.00 ppm.

Matrix assisted laser desorption/ionization Time-of-flight (MALDI-TOF). MALDI-TOF spectra were obtained on an OmniFlex MALDI-TOF mass spectrometer (Bruker Daltonics Inc.). The instrument was operated in a positive ion reflector mode with an accelerating potential of +20 kV. The TOF mass analyzed has a pulsed ion extraction. The linear flight path is 120 cm. OMNIFLEX TOF control software was used for hardware control and calibration. Spectra were obtained by averaging at least 300 laser shots. The pulsed ion extraction delay time was 200 ns. The spectrometer was calibrated using Angiotensin II as the external standard (1046.54 amu). To generate the matrix, a saturated solution of α-cyano-4-hydroxycinnamic acid (CCA) was prepared in a water:acetonitrile (2:1 v/v) with 0.1% TFA (TA solution). Oligopeptide samples dissolved in 10 μL DMSO with 0.1% TFA were diluted with 240 μL of TA solution so that the final concentration of oligopeptide was ˜10 pmol/μL. A 5 μL aliquot of this solution was mixed with 5 μL of CCA (matrix) solution in a 100 μL eppendorf tube. Then, 0.5 μL of this mixture was applied to the steel target that was then dried in ambient air. The abundance intensities of peaks at m/z values were collected via X-massOMNIFLEX6.0.0. software. Molecular weights obtained by experimental data were compared to a theoretical database created in MS Excel for the different end-capped peptides.

### Results

Conversions of γ-L-(Et)₂-Glu to oligo(γ-L-Et-Glu) as well as C-terminal functionalization is driven kinetically by using the activated Glu-di-ethyl eSter^{19,31}. Reactions are also thermodynamically driven towards oligomer formation by product precipitation when chain lengths reach DP_{avg}˜8.^{22,23,32} Hence, competing reactions due to hydrolysis in the aqueous reaction media are largely overcome. Scheme 2 shows these characteristics as well as the devised one-pot reaction to form C-terminal functionalized oligopeptide. It is apparent that potential pathways to products can occur by i) reaction between NH₂—R and γ-L-Et₂-Glu to form γ-L-Et-Glu-NH—R that subsequently propagates to form oligomeric product or ii) oligomerization of γ-L-(Et)₂-Glu to oligo(γ-L-Et-Glu)ₙ followed by reaction with NH₂—R to form oligo(γ-L-Et-Glu)ₙ-NH—R. The latter can precipitate or continue to propagate adding x γ-L-(Et)₂-Glu units prior to precipitation (forming oligo(γ-L-Et-Glu)ₙ₊ₓ-NH—R).

### Protease Characterization:

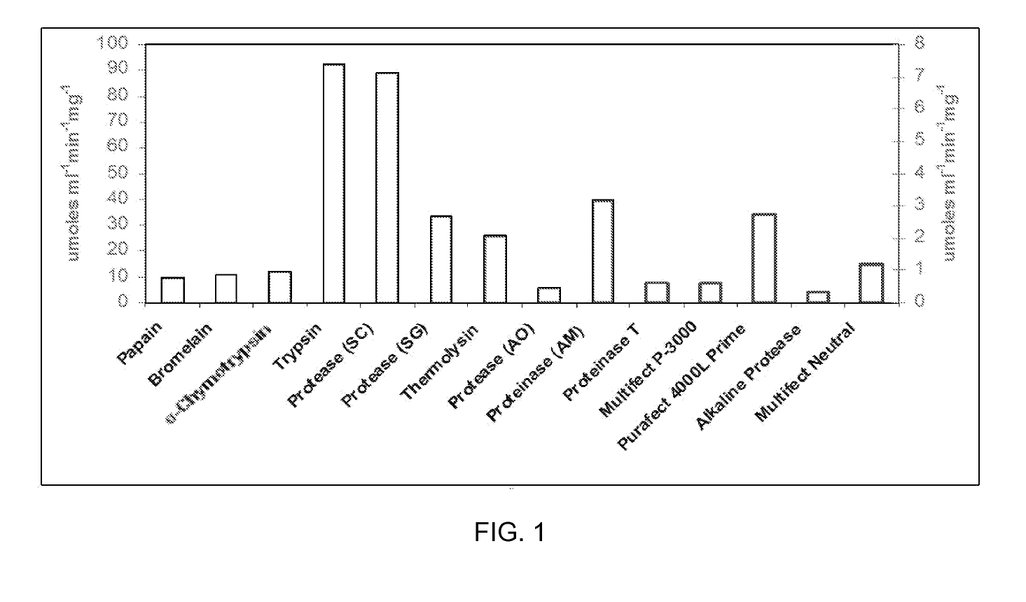

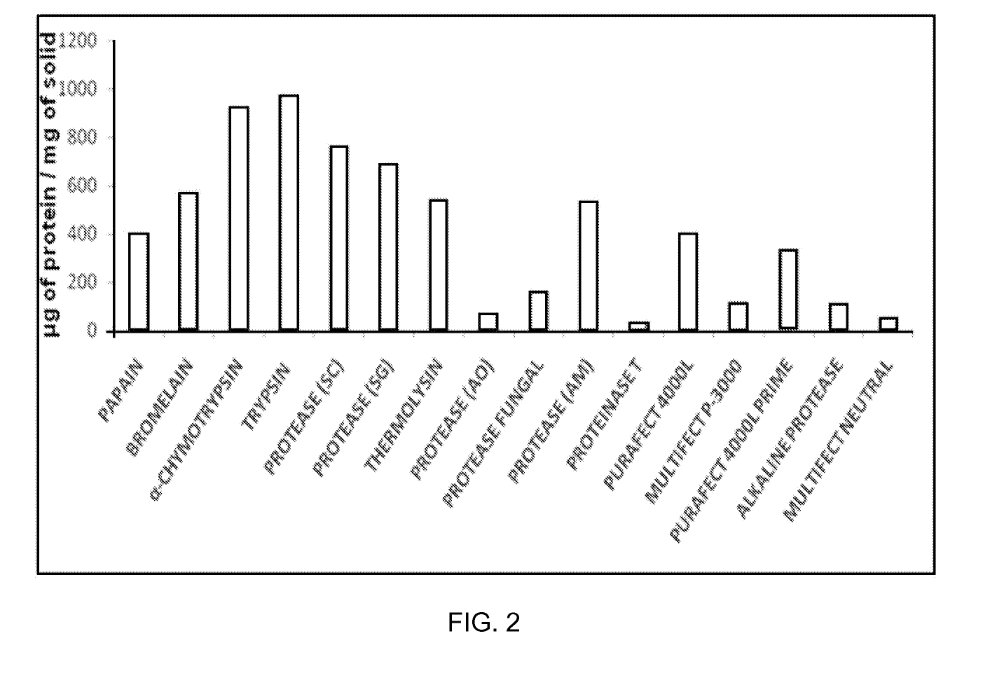

All proteases used herein were characterized to determine their hydrolytic activity by a casein assay and their protein content by the BCA method^{33,34} (see experimental). Values obtained from these measurements are shown in FIGS. 1 and 2, respectively. The amount of protein used in reactions was normalized based on casein hydrolysis activity. In summary, the protease samples, Multifect P-3000, Purafect prime 4000L and Alkaline protease had low protein content but very high (casein) hydrolytic activity. In contrast, α-chymotrypsin, Protease Sg and trypsin had high protein content and high hydrolytic activity while papain, bromelain, and Proteinase Am had low protein content and moderate hydrolytic activity. In all experiments that follow, 16 units/mL (a unit is the quantity of protease required to release 1 μmol of tyrosine equivalents per min per mL at pH 7.5 and 37° C.) of protease was used to catalyze oligo(γ-L-Et-Glu) synthesis and concurrent C-terminal functionalization with amine nucleophiles [oligo(γ-L-Et-Glu)-NH—R].

### Optimum pH for Oligopeptide Synthesis:

Li et al.²³ reported that optimal pH values for conversion of γ-L-(Et)₂-Glu to oligo(γ-L-Et-Glu) by papain, bromelain, α-chymotrypsin and Protease Sg are 6-8, 8-9, 9 and 8-9, respectively. Since %-yields of oligo(γ-L-Et-Glu) using papain, bromelain and α-chymotrypsin exceeded 30%, these three proteases were selected for experiments herein on concurrent oligo(γ-L-Et-Glu) synthesis and end-capping reactions.^{22,23} Along with the above enzymes, additional proteases were evaluated for use. Trypsin, Protease Sg, Protease Am, Multifect P-3000 and Purafect 4000L prime were assayed for oligo(γ-L-Et-Glu) synthesis at initial pH values of 7, 8, 9 and 10 (see FIG. 13). Only Multifect P-3000 and Purafect prime 4000L gave oligo(γ-L-Et-Glu) in yields >30%. Thus, in addition to papain, bromelain and α-chymotrypsin, Multifect P-3000 and Purafect prime 4000L were selected for C-terminal modification studies. Trypsin, Protease Sg, Protease Am gave oligo(γ-L-Et-Glu) yields up to 11% at initial pH=9. Thermolysin, Protease AO, Protease T and Purafect 4000L gave no precipitate suggesting that all these proteases were inactive for γ-L-(Et)₂-Glu oligomerization to chain lengths with DP_{avg}˜7. For both Multifect P-3000 and Purafect prime 4000L, increase in initial pH from 8 to 9 gave corresponding increases in product yield (25 to 41% and 17 to 36%, respectively).

With respect to hydrolytic activities, optimal pH values for trypsin, Protease Sg, Protease Am, Multifect P-3000 and Purafect prime 4000L occurs at 7-9, 5-9, 6-8, 7.5 and 6.5-10.5, respectively. In contrast, pH optima for oligo(γ-L-Et-Glu) synthesis with these proteases occurs at 7, 9, 9, 9 and 9 respectively²³. Similarly, papain, bromelain and α-chymotrypsin have optimum hydrolytic activity at pH 6-8, 4-8, 6-9, respectively, and optimum pH values for synthetic activity at 8, 8 and 9, respectively. Hence, with the exception of trypsin, high pH values within or above the range of protease activity for peptide bond hydrolysis were preferred for peptide synthesis.^{22,23,35,36}

These results are explained by considering that α-NH₂ moieties of amino acids have pKa value of 8.1-10.6. For γ-L-(Et)₂-Glu, the α-NH₂ pKa is ˜9.4. Therefore, by shifting the pH to values close to or above 9, an increase in the population of free or non-protonated NH₂ groups occurs thereby increasing their reactivity for accepting alkyl activated acyl groups. Accordingly, by the same principle, reactions between amine nucleophiles NH₂—R with γ-L-(Et)₂-Glu or propagating γ-L-Et-Glu oligomers should be enhanced at high pH values that favor a larger population of nucleophiles in the free amine form. Nevertheless, the reaction pH cannot be increased to a value that would result in protease denaturation thereby eliminating the three-dimensional protein structure that supports protease activity.

Overall, based on oligo(γ-L-Et-Glu) yields at optimal pH values for this synthetic reaction, the relative order of protease activities is as follows: papain-bromelain>α-chymotrypsin>Multifect P-3000>Purafect prime 4000L>Protease Sc˜Proteinase Am˜Protease Sg˜trypsin. Other proteases investigated appeared inactive for conversion of γ-L-(Et)₂-Glu to oligo(γ-L-Et-Glu) synthesis. The DP_{avg} of oligo(γ-L-Et-Glu) synthesized by these proteases was determined by ¹H-NMR (see discussion below). Oligo(γ-L-Et-Glu) DP_{avg} values were one or two units shorter for oligomers prepared by trypsin, Protease Sg and Proteinase Am (8.5, 8.3, 8.2, respectively) relative to those prepared by Multifect P-3000 and Purafect 4000L prime (9.2 and 9.5, respectively).

### Structural Analysis by ¹H NMR Spectroscopy:

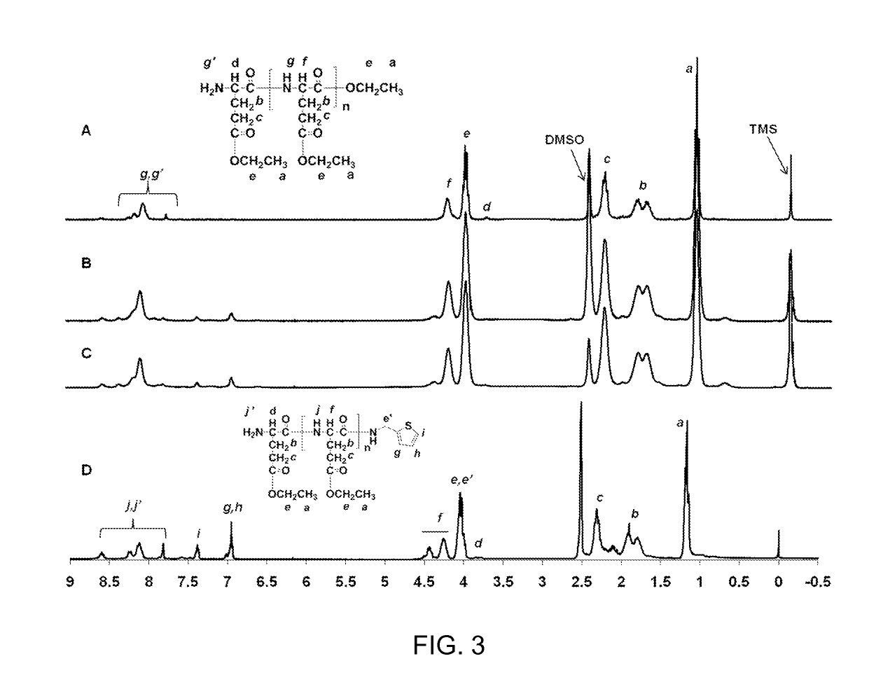

The spectrum for Multifect P-3000 catalyzed oligo(γ-L-Et-Glu) is shown in FIG. 3A. Peak assignments were based on published literature.^{19,22} Methine resonances of γ-L-Et-Glu repeat units (including those of C-terminal resonances), are found at 4.25 ppm (protons f), whereas N-terminal methine resonances (protons d) for γ-L-Et-Glu units are at ˜3.7 ppm. Hence, direct comparison of signal intensities at 4.3 and 3.7 ppm was used to determine DP_{avg} values of oligo(γ-L-Et-Glu). For Multifect P-3000 catalyzed reactions of TPMA with γ-L-(Et)₂-Glu, a shoulder with peaks in the region 4.3 to 4.45 ppm appeared corresponding to protons of C-terminal modified γ-L-(Et)-Glu repeat units. Relative integration of methine proton signals f at 4.25-4.45 and d at 3.7 ppm, respectively, was used to calculate DP_{avg}. Unless otherwise specified, protons f remain at ˜4.25-4.45 ppm when other nucleophiles in Scheme 1 replace TPMA at the C-terminal site of oligo(γ-L-Et-Glu). The percent C-terminal modification by reaction with NH₂—R nucleophiles in Scheme 1 was determined from the relative integration of protons corresponding to NH₂—R and the side chain protons of b corresponding to α-CH₂ of oligo(γ-L-Et-Glu).

Following the above mentioned protocol, the percent C-terminal modification by reaction with NH₂—R nucleophiles in Scheme 1 was determined from relative intensities of oligo(γ-Et-L-Glu) α-CH₂ side chain protons b and protons i, h and g between 6.9 and 7.5 ppm of the TPMA thiophene moiety. Assignments for ¹H NMR signals that were used to quantify other R—NH₂ groups used in this invention are listed above. ¹H NMR spectra in FIGS. 3B, 3C and 3D show how varying the protease used (papain, bromelain and Multifect P-3000, respectively) result in changes in efficiency of end-capping reactions. Reactions investigated used TPMA as the amine nucleophile and the ratio of TPMA-to-γ-L-Et₂-Glu in the monomer feed was maintained at 7:3. The discussion below relies upon ¹H NMR as above to quantify the extent that different NH₂—R nucleophiles react with ester groups (—[C═O]—O-Et) of either propagating oligo(γ-L-Et-Glu) chains or monomer (γ-L-Et₂-Glu).

Table 1 lists cumulative %-yields of precipitated oligo(γ-L-Et-Glu) chains with and without C-terminal end-capped oligo(γ-L-Et-Glu) chains as well as the percent of oligo(γ-L-Et-Glu) chains modified at the C-terminus with NH₂—R end-capping nucleophiles. Table 2 lists %-yield and %-oligo(γ-L-Et-Glu) modified at the ‘C’-terminus with R—NH₂ end-capping nucleophiles as a function of the γ-L-Et₂-Glu to R—NH₂ ratio, enzyme origin, and R—NH₂ structure. Table 3 lists %-yield and %-oligo(γ-L-Et-Glu) modified at the ‘N’-terminus with R—COOEt end-capping acyl donor as a function of the γ-L-Et₂-Glu to R—COOEt ratio, enzyme origin, and R—COOEt structure. The variables studied included the protease origin, ratio of γ-L-Et₂-Glu-to-NH₂—R in the monomer feed and NH₂—R structure. Reactions were performed in 0.9M sodium phosphate buffer solution at 40° C. for 3 h. Initial medium pH were adjusted to 8, 8, 9, 9 and 9 corresponding to pH optima values determined above for papain, bromelain, α-chymotrypsin, Multifect P3000 and Purafect prime 4000L, respectively. Without amine nucleophile in reactions, oligo(γ-L-Et-Glu) %-yield with the above proteases is 75±4%, 76±3% and 45±5%, 41±4% and 36±4%, respectively. Inspection of Table 1 shows that, for all five proteases and amine nucleophiles studied, addition of increased quantities of amine nucleophiles from 0 to 30, 50 and 70 mol % with respect to γ-L-Et₂-Glu results in decreased %-yield but increased mol % of [oligo(γ-L-Et-Glu)-NH—R]. These trends were intuitively expected since, an increase in the ratio of NH₂—R: γ-L-Et₂-Glu groups: i) can cause an increase in the number of chains formed thereby creating a fraction of reduced DP_{avg} oligo(γ-L-Et-Glu)-NH—R that does not precipitate; ii) results in a reduced concentration of oligomerizable substrate in reactions; and iii) NH₂—R might also function as a competitive inhibitor.

TABLE 1
%-Yield and %-oligo(γ-L-Et-Glu) modified at the ‘C’-terminus with
R—NH₂ end-capping nucleophiles as a function of the
γ-L-Et₂-Glu to R—NH₂ ratio, enzyme origin, and R—NH₂ structure.^{a,b}

| | | γ-L-Et₂-Glu to R—NH₂ nucleophile^{c} | | | | | |
|---|---|---|---|---|---|---|---|
| | | | | | 7:3 | 5:5 | 3:7 |
| R—NH₂ | | 7:3^{c} | 5:5 | 3:7 | % modified | | |
| Nucleophile | Enzyme | % yield^{d} | | | chains^{d} | | |
| TPMA | papain | 63 | 40 | 20 | 25 | 50 | 70 |
| | bromelain | 49 | 20 | 10 | 50 | 60 | >90 |
| | α-chymotrypsin | 18 | 10 | 5 | 75 | 0 | 0 |
| | Multifect P-3000 | 31 | 0 | 0 | >90 | 0 | 0 |
| | Purafect prime | 17 | 0 | 0 | >90 | 0 | 0 |
| | 4000L | | | | | | |
| TPEA | papain | 63 | 50 | 10 | 20 | 25 | 0 |
| | bromelain | 33 | 15 | 0 | 0 | 0 | 0 |
| | α-chymotrypsin | 23 | 10 | 0 | 0 | 0 | 0 |
| | Multifect P-3000 | 5 | 0 | 0 | 0 | 0 | 0 |
| | Purafect prime | 5 | 0 | 0 | 0 | 0 | 0 |
| | 4000L | | | | | | |
| FMA | papain | 50 | 45 | 15 | 15 | 20 | 30 |
| | bromelain | 45 | 40 | 10 | 25 | 40 | 45 |
| | α-chymotrypsin | 30 | 15 | 3 | 37 | 50 | 0 |
| | Multifect P-3000 | 20 | 0 | 0 | 42 | 0 | 0 |
| | Purafect prime | 3 | 0 | 0 | 50 | 0 | 0 |
| | 4000L | | | | | | |
| MFMA | papain | 47 | 27 | 0 | 20 | 44 | 0 |
| | bromelain | 36 | 0 | 0 | 22 | 0 | 0 |
| | α-chymotrypsin | 22 | 10 | 0 | 36 | 50 | 0 |
| | Multifect P-3000 | 0 | 0 | 0 | 0 | 0 | 0 |
| | Purafect prime | 0 | 0 | 0 | 0 | 0 | 0 |
| | 4000L | | | | | | |

^{a}reactions carried out in 0.9M phosphate buffer, for 3 h, at 40° C. and at the pH optimum for each enzyme used.
^{b}yield %-error was less than ±6%.
^{c}underlined number gives the value in the ratio of R—NH₂.
^{d}%-yield and %-modified chains were determined assuming oligopeptides consist of 8 γ-Et-Glu repeat units. For products containing ‘C’-terminal modified moieties [oligo(γ-L-Et-Glu)-NH—R], the %-yield was calculated by subtracting the weight contributed by ‘C’-terminal moieties as determined by ¹H NMR. Thus, %-yield is total product yield [oligo(γ- L-Et-Glu) + oligo(γ-L-Et-Glu)-NH—R] where the contribution of NH₂—R to product weight is subtracted.

TABLE 2
%-Yield and %-oligo(γ-L-Et-Glu) modified at the ‘C’-terminus
with R—NH₂ end-capping nucleophiles as a function of the
γ-L-Et₂-Glu to R—NH₂ ratio, enzyme origin, and R—NH₂ structure.^{a,b}

| | | γ-L-(Et)₂-Glu to R—NH₂ nucleophile^{c} | | | | | |
|---|---|---|---|---|---|---|---|
| | | | | | 7:3 | 5:5 | 3:7 |
| R—NH₂ | | 7:3 | 5:5 | 3:7 | % modified | | |
| Nucleophile | Enzyme | % yield^{d} | | | chains^{d} | | |
| BzA | papain | 45 | 30 | 25 | 20 | 46 | 55 |
| | bromelain | 40 | 38 | 20 | 36 | 55 | 62 |
| | α-chymotrypsin | 30 | 8 | 0 | 30 | 0 | 0 |
| | Multifect P-3000 | 0 | 0 | 0 | 0 | 0 | 0 |
| | Purafect prime | 0 | 0 | 0 | 0 | 0 | 0 |
| | 4000L | | | | | | |
| MPy | papain | 50 | 35 | 20 | 17 | 30 | 40 |
| | bromelain | 55 | 35 | 30 | 15 | 18 | 26 |
| | α-chymotrypsin | 30 | 7 | 0 | 40 | 0 | 0 |
| | Multifect P-3000 | 5 | 0 | 0 | 0 | 0 | 0 |
| | Purafect prime | 3 | 0 | 0 | 0 | 0 | 0 |
| | 4000L | | | | | | |
| NorbA | papain | 70 | 51 | np^{e} | 0 | 15 | np^{e} |
| | bromelain | 60 | 40 | np^{e} | 0 | 15 | np^{e} |
| | α-chymotrypsin | 45 | 30 | np^{e} | 0 | 18 | np^{e} |
| | Multifect P-3000 | 0 | 0 | 0 | 0 | 0 | 0 |
| | Purafect prime | 0 | 0 | 0 | 0 | 0 | 0 |
| | 4000L | | | | | | |
| F—OH | papain | 50 | 42 | 15 | 20 | 30 | 45 |
| | bromelain | 57 | 50 | 10 | 0 | 20 | 30 |
| | α-chymotrypsin | 40 | 20 | 0 | 30 | 40 | 0 |
| | Multifect P-3000 | 15 | 0 | 0 | 45 | 0 | 0 |
| | Purafect prime | 8 | 0 | 0 | 0 | 0 | 0 |
| | 4000L | | | | | | |

^{a}reactions carried out in 0.9M phosphate buffer, for 3 h, at 40° C. and at the pH optimum for each enzyme used.
^{b}yield %-error was less than ±6%.
^{c}underlined number gives the value in the ratio of R—NH₂.
^{d}%-yield and %-modified chains were determined assuming oligopeptides consist of 8 γ-Et-Glu repeat units. For products containing ‘C’-terminal modified moieties [oligo(γ-L-Et-Glu)-NH—R], the %-yield was calculated by subtracting the weight contributed by ‘C’-terminal moieties as determined by ¹H NMR. Thus, %-yield is total product yield [oligo (γ-L-Et-Glu) + oligo(γ-L-Et-Glu)-NH—R] where the contribution of NH₂—R to product weight is subtracted.
^{e}np is an abbreviation for reaction not performed.

TABLE 3
%-Yield and %-oligo(γ-L-Et-Glu) modified at the ‘N’-terminus with
R—COOEt end-capping acyl donor as a function of the γ-L-Et₂-Glu to
R—COOEt ratio, enzyme origin, and R—COOEt structure.^{a,b}

| | | γ-L-(Et)₂-Glu to R—COOEt acyl donor^{c} | |
|---|---|---|---|
| R—COOEt | | 7:3 | 7:3 |
| Acyl donor | Enzyme | % yield^{d} | % modified chains^{d} |
| AcE | papain | 33 | 60 |
| | bromelain | 35 | 0 |
| | α-chymotrypsin | 10 | 0 |
| | Multifect P-3000 | 0 | 0 |
| | Purafect prime | 0 | 0 |
| | 4000L | | |
| AcF | papain | 45 | 30 |
| | bromelain | 25 | 25 |
| | α-chymotrypsin | 0 | 0 |
| | Multifect P-3000 | 0 | 0 |
| | Purafect prime | 0 | 0 |
| | 4000L | | |

^{a}reactions carried out in 0.9M phosphate buffer, for 3 h, at 40° C. and at the pH optimum for each enzyme used.
^{b}yield %-error was less than ±6%.
^{c}underlined number gives the value in the ratio of R—NH₂.
^{d}%-yield and %-modified chains were determined assuming oligopeptides consist of 8 γ-Et-Glu repeat units. For products containing ‘C’-terminal modified moieties [oligo(γ-L-Et-Glu)-NH—R], the %-yield was calculated by subtracting the weight contributed by ‘C’-terminal moieties as determined by ¹H NMR. Thus, %-yield is total product yield [oligo(γ-L-Et-Glu) + oligo(γ-L-Et-Glu)-NH—R] where the contribution of NH₂—R to product weight is subtracted.

Of the different NH₂—R compounds evaluated, TPMA gave the highest fraction of oligo(γ-L-Et-Glu)-NH—R chains in the precipitated product. Intriguingly, this result is true regardless of the protease used. However, distinct differences were found in the capability of different proteases to form oligo(γ-L-Et-Glu)-TPMA. For instance, with γ-L-Et₂-Glu to TPMA of 7:3, the mol % of oligo(γ-L-Et-Glu)-TPMA formed with papain, bromelain, α-chymotrypsin, Multifect P-3000 and Purafect prime 4000L is 25, 50, 75, >95 and >95, respectively. Also, using bromelain catalysis and ratios of Et₂-Glu to TPMA of 5:5 and 3:7, the mol-% of oligo(γ-L-Et-Glu)-TMPA formed were 80 and >90%, respectively. For Multifect P-3000, the attainment of >95 mol-% of chains with TPMA C-terminal groups is particularly exciting since the decrease in product yield relative to 0 mol % TPMA in reactions was only 10% (41 to 31%).

Given the high reactivity of TPMA for protease-catalyzed C-terminal end-capping, experiments were performed to explore structural analogs of TPMA. The goal was to derive insights into protease promiscuity and, thereby, an understanding of the range of related amine nucleophiles that may be incorporated at oligo(γ-L-Et-Glu) C-termini. TPMA and TPEA differ structurally by one methylene unit between the thiophene and amine groups (see Table 1). Table 1 shows that, of the five proteases studied, only papain formed oligo(γ-L-Et-Glu)-TPEA chains. For example the reaction catalyzed by papain with γ-L-Et₂-Glu-to-TPEA 5:5 mol/mol, 25 mol-% of precipitated product consisted of oligo(γ-L-Et-Glu)-TPEA. Multifect P-3000 and Purafect prime 4000L, which gave >90 mol % of oligo(γ-Et-Glu)-TPMA with γ-L-Et₂-Glu to TPEA 7:3, was ineffective in forming oligo(γ-L-Et-Glu)-TPEA. This large difference in reactivity between TPMA and TPEA, found for all five proteases studied, can be explained by that TPMA more closely resembles α-amino acids.

A close analog of TPMA is FMA which contains a furan in place of the thiophene ring. Comparison of total product yield [oligo(γ-L-Et-Glu)+oligo(γ-L-Et-Glu)-FMA] obtained as a function of the γ-L-Et₂-Glu-to-FMA ratio in the monomer feed was generally similar. However, for all the proteases used, the mol % of oligo(γ-L-Et-Glu)-FMA chains was clearly less than what was obtained using TPMA as NH₂—R. For example, with Et₂-Glu to FMA of 7:3, the mol-% oligo(γ-L-Et-Glu)-FMA formed with papain, bromelain, α-chymotrypsin, Multifect P-3000 and Purafect prime 4000L is 15, 25, 37, 42 and 50%, respectively. In comparison, using TPMA, the mol % of oligo(γ-L-Et-Glu)-TPMA formed was 25, 50, 75, >90 and >90, respectively. Therefore, substitution of thiophene with a furan ring had a large effect on NH₂—R efficiency.

FMA and MFMA differ by a methyl substituent at the furan ring 5-position. Comparison of total product yield [oligo(γ-L-Et-Glu)+oligo(γ-L-Et-Glu)-MFMA]obtained as a function of γ-L-Et₂-Glu to MFMA showed that, for all 5 proteases, yields were lower when using MFMA instead of FMA as NH₂—R. For example, for γ-L-Et₂-Glu to NH₂—R 5:5 and using bromelain as catalyst, product yield was 40 and 0% with FMA and MFMA, respectively. When using α-chymotrypsin, Multifect P-3000 and Purafect prime 4000L, mol %-incorporation of FMA and MFMA at C-termini were similar. In contrast, with papain as catalyst and γ-L-Et₂-Glu to NH₂—R of 5:5, incorporation of MFMA was higher than FMA (44 vs 20%). As discussed above, lower product yields obtained with MFMA may be due to an increase in the number of chains formed resulting in a fraction of oligo(γ-L-Et-Glu)-NH—R that does not precipitate or to its function as a competitive inhibitor.

Results shows that, for all five proteases and amine nucleophiles studied, addition of increased quantities of amine nucleophiles from 0 to 30, 50 and 70 mol % with respect to Et₂-Glu, resulted in decreased %-yield of precipitated product but increased contents of oligo(γ-L-Et-Glu)-NH—R. Also, the extent of decreased yield and increased end-capping was highly sensitive to the protease-NH₂—R pair studied. Of interest was to explore amino acids as C-terminal end-capping compounds where the acid moiety is non-activated by esterification (e.g. in free acid or salt form).^{30,37} Given its importance in metal binding, L-histidine (H) was studied as an amine nucleophile during oligo(γ-L-Et-Glu) synthesis. Using papain, bromelain, and α-chymotrypsin as protease-catalysts and a feed ratio of 5:5 γ-L-Et₂-Glu/H, total %-product yields obtained were 40, 45 and 15%, respectively. ¹H-NMR and MALDI-TOF spectra for L-histidine end-capping experiments showed an absence of peaks corresponding to incorporation of H at oligo(γ-L-Et-Glu) C-termini. Furthermore, when H was employed as amine nucleophile using Multifect P-3000 and Purafect prime 4000L as catalysts, no precipitate was obtained at all feed ratios studied. Furthermore, for all five proteases studied herein, L-phenylalanine (F) addition to γ-L-Et₂-Glu did not produce end-capped oligo(γ-L-Et-Glu)-F. Interestingly, previous attempts documented in the literature to use inactivated F and H as amine nucleophiles gave low product yields. Specifically, carboxypeptidase-Y catalyzed reaction of Bz-Ala-(C═O)—OMe with F and H gave dipeptide in 25 and 15% yield, respectively. Furthermore, thermolysine catalyzed coupling of H₂N—(O═C)-His-NH₂ (α-carboxylamide derivative of H) with Z-Phe-OH resulted in no dipeptide formation.³⁸

In an effort to understand what structural features of these natural amino acids resulted in their inability to react as amine nucleophiles during γ-L-Et₂-Glu synthesis, further experiments were performed with both F and H analogs. L-Phenylmethanamine (BzA) is similar to F but lacks an α-carboxyl group. For γ-L-Et₂-Glu to BzA 7:3, the mol % of oligo(γ-L-Et-Glu)-BzA formed with papain, bromelain and α-chymotrypsin is 20, 36, 30%, respectively. When using Multifect P-3000 and Purafect prime 4000L as catalysts, no precipitated product was formed. By increasing the molar ratio of γ-L-Et₂-Glu-to-BzA to 3:7, the mol % of oligo(γ-L-Et-Glu)-BzA with papain and bromelain increased to 55 and 62%, respectively. Thus, relative to TPMA, BzA was not as effective in forming end-group modified products. However, substitution of the α-carboxyl group of F with a hydrogen atom was found to be beneficial to enabling BzA to function as a C-terminal end-capping group during synthesis of oligo(γ-L-Et-Glu) (there being a difference of one methylene unit between the structures of BzA and phenylalanine).

A close analog of BzA is 4-methylamino pyridine (MPy) which contains a pyridine ring in place of the phenyl ring. Comparison of total product yield obtained as a function of γ-L-Et₂-Glu to MPy was generally similar. The relative efficiency of BzA and MPy end-capping of oligo(γ-L-Et-Glu) chains during oligomer synthesis changed as a function of the protease used. For papain and bromelain, incorporation of BzA was higher than MPy. However, for γ-L-Et₂-Glu to NH₂—R 7:3, α-chymotrypsin gave higher contents of MPy end-groups. As observed above with thiophene and furan ring-structures, and here with pyridine all of which are not found in naturally occurring amino acids, the promiscuity of proteases to incorporate these non-natural structures as C-terminal groups is useful in diversifying the end-group structure and, therefore, potential functions of synthesized oligopeptides.

To further investigate the potential that it is the α-carboxyl group of F and H that results in their inability to function as amine nucleophiles for oligo(γ-L-Et-Glu) end-capping, L-phenylalaminol (F—OH) (Scheme 1) was studied. F—OH is the reduced form of L-phenylalanine so that —CH₂—OH replaces the anionic α-carboxyl group. Whereas L-phenylalanine addition to L-Et₂-Glu did not produce end-capped oligo(γ-L-Et-Glu), for all five proteases studied herein, addition of F—OH to γ-L-Et₂-Glu in the monomer feed gave substantial quantities of oligo(γ-L-Et-Glu)-F—OH chains. Also, total precipitated product for γ-L-Et₂-Glu to L-phenylalaminol 7:3 remained high relative to total product yields reported for other NH₂—R compounds in Tables 1 and 2. Multifect P-3000 was particularly active in forming end-capped chains with Et₂-Glu to L-phenylalaminol (F—OH) 7:3. However, further increase in the γ-L-Et₂-Glu to L-phenylalaminol ratio to 5:5 for Multifect P-3000, and to 3:7 for α-chymotrypsin, resulted in no precipitated product. Hence, in these cases, F—OH was either a strong inhibitor of peptide synthesis or formed low-molecular weight end-capped chains that did not precipitate. Indeed, earlier literature describes that peptide sequences bearing terminal hetero-aromatic moieties can act as potent protease inhibitors.^{39,40}

A series of experiments was performed using bicyclo[2.2.1]-5-heptene-2-methylamine (NorbA) as the amine nucleophile during oligo(γ-L-Et-Glu) synthesis. NorbA was synthesized by reduction of bicyclo[2.2.1]-5-heptene-2-carbonitrile following a literature procedure.⁴¹ Using papain, bromelain, and α-chymotrypsin as protease catalysts and a γ-L-Et₂-Glu-to-NorbA ratio of 5:5 gave total product yields of 51, 40 and 30%, respectively. Analysis by ¹H NMR showed that the mol-% of oligo(γ-L-Et-Glu)-NH-NorbA chains were 15, 15 and 18%, respectively. In contrast to papain, bromelain and α-chymotrypsin, Multifect P-3000 and Purafect prime 4000L yielded no product even when the ratio of γ-L-Et₂-Glu-to-NorbA in the monomer feed was 7:3. The relatively low level of oligo(γ-L-Et-Glu)-NH-NorbA formed during oligopeptide synthesis compared to when other NH₂—R nucleophiles such as TPMA, FMA, MFMA, MPy and BzA and F—OH were used is attributed to the bulky structure of the bycyclic NorbA ring structure.

### MALDI-TOF Analysis of End Capped Products:

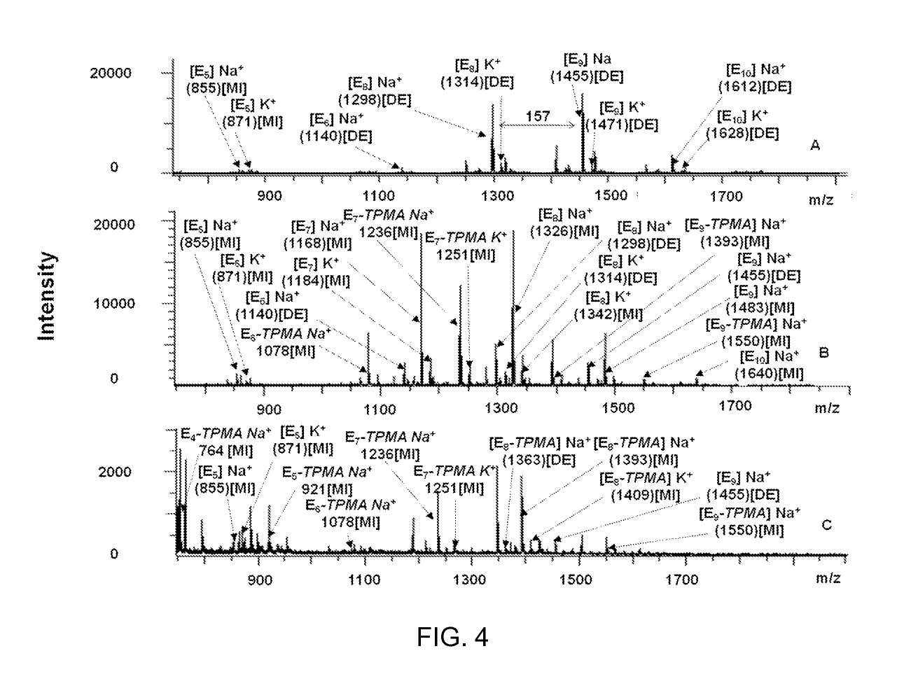

Oligo(γ-L-Et-Glu) catalyzed by Multifect P-3000 without addition of an R—NH₂ nucleophile produces a series of signals in MALDI-TOF spectrum as shown in FIG. 4A corresponding to oligo(γ-L-Et-Glu)[Eₙ] where ‘n’ represents the number of repeat units. For example, signals are observed at m/z 855 and 871, corresponding to the E₅ molecular ion peak associated with a sodium and potassium, respectively. In addition, signals corresponding to E₆ (1140), E₈ (1298, 1314), E₉ (1455, 1471) and E₁₀ (1612,1628) corresponds to (γ-L-Et-Glu) with one de-esterified γ-L-Glu unit, associated with a sodium and potassium ions, respectively. The highest intensity signals in MALDI-TOF spectrum of FIG. 4A are observed for E₈ and E₉. This is in excellent agreement with DP_{avg} 9.5 determined by ¹H NMR for Multifect P-3000 catalyzed oligo(γ-L-Et-Glu) synthesis.

Spectrum B of FIG. 4B was recorded of products synthesized by bromelain catalysis using a 7:3 molar ratio of L-Et₂-Glu-to-TPMA. Based on ¹H NMR, the product has a DP_{avg} of 8.3 and consists of a 50:50 mixture of oligo(γ-L-Et-Glu)-NH-TPMA and oligo(γ-L-Et-Glu). As anticipated, the MALDI-TOF spectrum in FIG. 4B shows identical signals that were listed and assigned above corresponding to oligo(γ-L-Et-Glu). The next set of peaks corresponding to oligo(γ-L-Et-Glu)-NH-TPMA molecular ion plus sodium are as follows: E₆ (1078), E₇ (1236), E₈ (1393), E₆ (1550).

Based on qualitative interpretation of the MALDI-TOF where the intensities of peaks at 1100 to 1350, 1400 to 1550, and 1600 to 1750 were compared, the percent oligo(γ-L-Et-Glu)-NH-TPMA to oligo(γ-L-Et-Glu) is 40±10%. Thus, the extent of end-group capping with TPMA catalyzed by bromelain using a 7:3 molar ratio of γ-L-Et₂-Glu-to-TPMA, determined by ¹H NMR, is in excellent agreement with the MALDI-TOF results. Spectrum C of FIG. 4C was recorded of products synthesized by Multifect P-3000 catalysis using a 7:3 molar ratio of L-Et₂-Glu-to-TPMA. Based on ¹H NMR, the product has a DP_{avg} of 9.3 and consists of >95% of oligo(γ-L-Et-Glu)-NH-TPMA. Consistent with the ¹H NMR analysis, the MALDI-TOF spectrum of FIG. 4C shows only low intensity signals corresponding to oligo(γ-Et-Glu) E₅ (855, 871) and E₉ (1455) with one ester group de-estrified and associated with sodium and potassium ions, respectively. MALDI TOF signals in FIG. 4C corresponding to the molecular ion of oligo(γ-L-Et-Glu)-NH-TPMA associated with sodium or potassium ion are at E₆ (1078,1094), E₇ (1236,1251), E₈ (1393,1409) and E₉ (1550). Based on qualitative interpretation of the MALDI-TOF spectrum in FIG. 4C, where the intensities of peaks at 800 to 1000, 1100 to 1300, and 1400 to 1700 were compared, the percent of end-functionalized oligo(γ-L-Et-Glu)-NH-TPMA chains in the total product is 90±5%. Hence, ¹H NMR and MALDI-TOF values of end-group capping with TPMA, catalyzed by Multifect P-3000, using a 7:3 molar ratio of γ-L-Et₂-Glu-to-TPMA, are in excellent agreement. The absence of low intensity peaks corresponding to one de-esterified ester group for oligo(γ-L-Et-Glu)-NH-TPMA in FIG. 4C suggests that de-esterification occurs at the C-terminal oligo(γ-L-Et-Glu) unit, not at other sites along the oligomer. In other words, if protease-catalyzed de-esterification occurred at sites other than the C-terminal unit, then nearly quantitative end-capping at this position could occur along with de-esterication giving the corresponding molecular ions in the MALDI-TOF spectrum.

### Illustrative Processes.

The present invention can be carried out in a number of ways. A first embodiment is a process for preparing oligopeptides end-functionalized at the N-terminus, C-terminus or at both ends that has the general formula oligomer of the formula CA-(AA)ₙ-B wherein B is a group at the carboxyl terminus, CA is a group at the N-terminus, AA is an amino acid and n is the oligomer chain length which comprises the steps of:
a) admixing one or more amino acid alkyl esters with one or more an end-functionalizing agents and one or more proteases in a reaction medium;
b) heating the mixture to between about 5° C. to about 90° C. for between 5 minutes and 24 hours; and
c) recovering the end-functionalized oligopeptide.

An embodiment of the process employs an amino acid alkyl ester having the following general formula (1):

H₂N—CH(R)—(CR′H)ₙ—COOX (1)

in which R represents an amino acid side chain, R′ represents a different amino acid side chain present in β-amino acids, and X is an alkyl ester preferably consisting of an alkyl group selected from those containing from one to six carbon atoms but may consist of up to 20 carbon atoms. The alkyl ester may be straight or branched chain and include methyl, ethyl, propyl, isopropyl, butyl, hexyl and the like. In a preferred embodiment, the alkyl ester is selected from the group consisting of methyl, ethyl or propyl groups. Activated esters can also be used in place of alkyl esters, and examples of activated esters include guanadinophenyl, p-nitrophenyl, 1,1,1,3,3,3-hexafluoroisopropyl, 2,2,2-triifluoroethyl, 2-chloro ethyl ester, carbamoyl methyl ester, benzyl esters, and anilides. Another embodiment of the process is where in general formula (1) n=0, the stereochemistry is L, and R is selected from one or more of the natural amino acid side chains shown in Chart 1.

Another embodiment of the process employs an amino acid alkyl ester having the following general formula (2):

H₂NCH₂CH(R)—COOX (2)

wherein the β-amino acids and other non-natural amino acid structures are those known to those of ordinary skill in the art as useful substrates for protease-catalyzed oligopeptide synthesis or protease-catalyzed coupling of preformed segments of oligo(amino acids). Illustrative β-amino acids are those that consist of the general structure (2) and have R groups selected from the above Chart 1. Examples of β-amino acids include: β-alanine, L-β-homotyrosine, β-homoleucine, L-β-homoisoleucine and L-β-homotryptophan. Examples of other non-natural amino acid esters include: carnitine [3-Hydroxy-4-trimethylammonio-butanoate], ornithine [(+)-(S)-2,5-diamino valeric acid], citruline [2-Amino-5-(carbamoylamino)pentanoic acid], 4-aminobutanoic acid and L-Dopamine, and other non-natural amino acids.

The most preferable alkyl ester group X in general formula (1) is ethyl.

CHART 1
Protein Amino Acids and Their Functional (R—) Groups
With Their 3-Letter & (1-Letter) Abbreviations

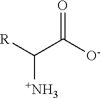

Neutral amino acids

| | |
|---|---|
| Glycine | H— |
| Gly (G) | |
| Alanine | H₃C— |
| Ala (A) | |
| Valine Val (V) | 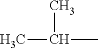 |
| Leucine Leu (L) | 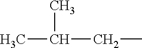 |
| Isoleucine Ile (I) | 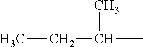 |
| Serine | HO—CH₂— |
| Ser (S) | |
| Threonine Thr (T) | 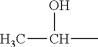 |

Sulfur amino acids

| | |
|---|---|
| Cysteine | HS—CH₂— |
| Cys (C) | |
| Methionine | H₃C—S—CH₂—CH₂— |
| Met (M) | |

Cyclic amino acids

| | |
|---|---|
| Proline Pro (P) | 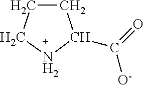 |

Aromatic amino acids

| | |
|---|---|
| Phenylalanine Phe (F) | 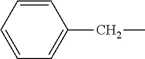 |
| Tyrosine Tyr (Y) | 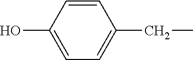 |
| Tryptophan Trp (W) | 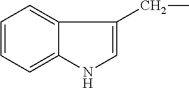 |
| Histidine His (H) | 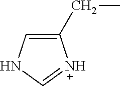 |

Basic amino acids

| | |
|---|---|
| Lysine Lys (K) | |
| Arginine Arg (R) | 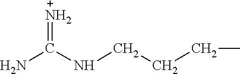 |

Acidic amino acids & their amides

| | |
|---|---|
| Aspartic acid Asp (D) | 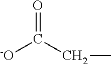 |
| Asparagine Asn (N) | 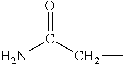 |
| Glutamic acid Glu (E) | 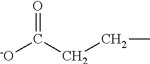 |
| Glutamine Gln (Q) | 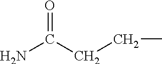 |

The reaction medium used can consist of a phosphate, acetate, borate, carbonate, HEPES, or sulphate buffers with concentrations that can vary widely but generally are between 0.1M to 1.5M. or, instead of buffer salts, some amines such as triethyl amine can be used to maintain reaction medium pH. A water-miscible cosolvent selected from the group consisting of formamides, alcohols (1°, 2°, 3°), dimethyl sulfoxide, tetrahydrofuran, acetone, acetonitrile, 1,2-ethylene glycol, 1,3-propylene glycol, or 1,4-butanediol can be added in concentrations from 0 to 50%-v/v.

In another embodiment, the enzyme or enzyme mixture is selected from a member of a hydrolytic enzyme family that is further comprised of proteases, lipases, esterases and cutinases. The enzyme can be selected from members of the protease family, and wherein:

(i) suitable proteases for use in this invention include papain, bromelain, α-chymotrypsin, trypsin, Multifect P-3000 (Genencor), Purafect prime L (Genencor), alkaline protease (Genencor), metalloprotease (thermolysin), protease from subtilisin (family), pronasel, glutaminase, carboxypeptidase Y, clostrapin, protease from aspergillus oryzae species, pepsin, cathepsin, ficin, alcalase, carboxypeptidase, calpains, actinidin, chymosin, carbonic anhydrase, nonribosomal peptide synthetase, thrombin, cardosins A or B or pronase;
(ii) a reaction can be catalyzed by one or a mixture of 2 or more proteases;
(iii) variants of these enzymes, generated by standard protein engineering methods such as error-prone PCR and gene shuffling, well known to those of ordinary skill in the art, can be used to further improve a proteases activity and selectivity for use in the current invention; and
(iv) suitable enzymes may be identified by other methods known by those skilled in the art, can be identified via searches of gene data banks, can subsequently be synthesized by preparation of the gene, cloning of the gene into a suitable host, and production of the enzyme by fermentation, and may be identified DNA mining from various environments such as in soil.
The enzymes can be added to the reaction media as enzyme powders, in solution, or immobilized on a support.

The reaction can be terminated in various manners. For example, the reaction can be terminated by filtration of the immobilized enzyme. The reaction also can be terminated by separation of the precipitated end-functionalized oligopeptide product by filtration or centrifugation from the enzyme remaining in the reaction medium. The reaction also can be terminated by using a membrane with a suitable pore size that separates a soluble end-functionalized oligopeptide product from the soluble enzyme. The reaction can be terminated by selective precipitation of either the soluble enzyme or the soluble end-functionalized oligopeptide product.

The reaction time preferably is between 5 minutes and 24 hours. More preferably, the reaction time is between 10 minutes and 8 hours. Even more preferably, the reaction time is between 30 minutes and 3 hours.

The reaction temperature is between 5° C. and 90° C. More preferably, the reaction temperature is between 25° C. and 60° C. Even more preferably, the reaction temperature is between 30° C. and 40° C.

In another embodiment of the invention, the reaction is performed by passing reactants through a column wherein the stationary phase consists of the immobilized enzyme.

The end-capped oligopeptides can consist of a mixture of oligomers. For example, the end-capped oligopeptides can consist of a mixture of oligomers where the average chain length, determined by measuring the number average molecular weight, ranges from 2 to 100 units. Preferably, the end-capped oligopeptides consist of a mixture of oligomers where the average chain length, determined by measuring the number average molecular weight, ranges from 5 to 50 units. More preferably, the end-capped oligopeptides consist of a mixture of oligomers where the average chain length, determined by measuring the number average molecular weight, ranges from 10 to 20 units.

Additionally, the end-capped oligopeptides can consist of a mixture of oligomers having a certain polydispersity. For example, the end-capped oligopeptides consist of a mixture of oligomers with a polydispersity, determined by dividing the weight average molecular weight by the number average molecular weight, of 50. Preferably, the end-capped oligopeptides consist of a mixture of oligomers with a polydispersity, determined by dividing the weight average molecular weight by the number average molecular weight, that is <25. More preferably, the end-capped oligopeptides consist of a mixture of oligomers with a polydispersity, determined by dividing the weight average molecular weight by the number average molecular weight, that is <5. Even more preferably, the end-capped oligopeptides consist of a mixture of oligomers with a polydispersity, determined by dividing the weight average molecular weight by the number average molecular weight, that is <1.5.

The end-functionalizing agent can consist of a member selected from the family of primary amines. Preferably, the end-functionalizing agent can be selected from a primary amine having the structure R—(CH₂)ₙ—NH₂, where R belongs to a member of the family of 5 or 6 membered rings. In one embodiment, R can be an aromatic 5 or 6 membered ring. For example, R can be an aromatic heterocyclic 5 or 6 membered ring that contains a sulfur, oxygen or nitrogen atom. For another example, R can be a phenyl ring with one or more substituents that can be selected from the group consisting of: —N₃, —NO₂, —OH, —F, —CI, —I, —COON, —CH₂═CH, —CH≡C—H. In a specific example, n is either 1 or 2.

The end-functionalization agent alternatively can consist of an activated ester with the general formula:

Y′Y—[H]ₐN—CH(R)—(CR′H)ₙ—COOX (3)

wherein X is an alkyl ester preferably consisting of an alkyl group selected from those containing from one to six carbon atoms but may consist of up to 20 carbon atoms, wherein (i) the alkyl ester may be straight or branched chain and include methyl, ethyl, propyl, isopropyl, butyl, hexyl and the like, (ii) preferably the alkyl ester is selected from the group consisting of methyl, ethyl or propyl groups, and (iii) activated esters can also be used in place of alkyl esters, (iv) examples of activated esters include guanadinophenyl, p-nitrophenyl, 1,1,1,3,3,3-hexafluoroisopropyl, 2,2,2-triifluoroethyl, 2-chloro ethyl ester, carbamoyl methyl ester, benzyl esters, and anilides.

Y and/or Y′ can be selected from H, methyl, ethyl, CH₂═CH—CO—, CH₂═C(CH₃)—CO—, HOOC—CH═CH—CO— (cis or trans) and other polymerizable groups known to one skilled in the art and wherein polymerizable groups may require a wide variety of polymerization methods known to one skilled in the art such as conventional free radical polymerization, ATRP, RAFT, and ring-opening metathesis reactions. Y and/or Y′ also can be selected from functional groups used for photolytic crosslinking that are well known to one skilled in the art, illustrative examples of which include a cinnamoyl (Ph-CH═CH—CO—) group. Y and/or Y′ also can be selected from structures well known to those skilled in the art that are crosslinkable via redox catalysts that may be of chemical or enzyme (e.g. laccases, peroxidases) origin, illustrative examples of which include HO-Ph-(CH₂)—CO— where the hydroxyl group is at the para-position. In one illustrative embodiment, n=0, R is selected from a member of the natural 20 amino acid side chains shown in Chart 1, Y is H, and Y′ is CH₂═CH—CO— or CH₂═C(CH₃)—CO—.

Another example process of the present invention is a process for preparing oligopeptides end-functionalized at the N-terminus, C-terminus or at both ends that has the general formula C-(AA)ₙ-B wherein B is a group at the carboxyl terminus, C is a group at the N-terminus, AA is an amino acid and n is the oligomer chain length which comprises the steps of:
a) admixing one or more amino acid alkyl esters one or more proteases in a reaction medium;
b) heating the mixture to between about 5° C. to about 90° C. for between 5 minutes and 24 hours;
c) recovering the oligopeptide; and
d) performing a modification of the N-terminal amino group by conventional coupling methods using conventional chemical methods well known by persons of ordinary skill in the art.

In this embodiment of the process, the N-terminal group of oligopeptides can be modified by N-acylation chemistry using conventional chemical methods well known by persons of ordinary skill in the art. Further, the N-acylated amino acids formed can have the following general structure:

R(C═O)NH[AA]ₙCOOX (4)

wherein R(C═O) can be derived from any of the following natural fatty acids:

| | Carbon | Double | | Common |
|---|---|---|---|---|
| Common Name | Atoms | Bonds | Scientific Name | Sources |
| lauric acid | 12 | 0 | dodecanoic acid | coconut oil |
| (LA) | | | | |
| myristic acid | 14 | 0 | tetradecanoic acid | palm kernel |
| (MA) | | | | oil |
| palmitic acid | 16 | 0 | hexadecanoic acid | palm oil |
| (PA) | | | | |
| palmitoleic | 16 | 1 | 9-hexadecenoic acid | animal fats |
| acid (POA) | | | | |
| stearic acid (SA) | 18 | 0 | octadecanoic acid | animal fats |
| oleic acid (OA) | 18 | 1 | 9-octadecenoic acid | olive oil |
| ricinoleic acid | 18 | 1 | 12-hydroxy-9- | castor oil |
| (RA) | | | octadecenoic acid | |
| linoleic acid (LA) | 18 | 2 | 9,12-octadeca- | grape seed |
| | | | dienoic acid | oil |
| α-linolenic | 18 | 3 | 9,12,15-octadeca- | flaxseed oil |
| acid (ALA) | | | trienoic acid | (linseed oil) |
| γ-linolenic | 18 | 3 | 6,9,12-octadeca- | borage oil |
| acid (GLA) | | | trienoic acid | |
| behenic acid (BA) | 22 | 0 | docosanoic acid | rapeseed oil |
| erucic acid (EA) | 22 | 1 | 13-docosenoic acid | rapeseed oil |

The fatty acid can be first modified by hydrogenation, epoxidation, hydroxylation, or any other method known by those skilled in the art prior to reaction with NH₂ terminal groups of oligopeptides. In additional embodiments, R can be selected from the group consisting of —CH₂═CH—CO—, CH₂═C(CH₃)—CO—, HOOC—CH═CH—CO— (cis or trans), and other polymerizable groups known to one skilled in the art. In further embodiments, the amino acids can consist primarily of glutamic acid units (>50 mol % of repeat units) and the other 50 mol % of repeat units can be selected from one or a mixture of two or more naturally occurring amino acids.

Following are additional examples of acids that could be potential N-terminal end capping reagents both for in-situ end-capping or post synthetic modification of oligoglutamic acid (produced by protease catalysis) to give lipopeptides.

Synthesis of C-terminal end capped oligoglutamate: The method used is identical to that for synthesis of oligo(γ-Et-L-Glu) except for the following modifications. Although the stoichiometry of L-(Et)₂-Glu.HCl and end-capping substrate was varied, the total concentration of monomer and end-capping substrate remained at 2.5 mmol. Due to the high basicity/acidity of end-capping nucleophiles, sodium phosphate buffer solution (5 mL, 0.9M) was added as above and solutions were titrated back to the desired pH using 10M NaOH or 10M HCl. Subsequently, 16 units/mL of the desired enzyme was added to catalyze oligopeptide synthesis. Precipitate formed was washed with 2×5 mL of de-ionized water then with an HCl solution (pH 2, 2×5 mL), separation of precipitate after each washing step was by centrifugation (5000 rpm), supernatants were discarded and the precipitate was freeze dried to obtain the product. The %-yield of amine end-capped chains of oligo(γ-L-Et-Glu) [oligo(γ-L-Et-Glu)-NH—R] was calculated gravimetrically from the precipitated product obtained.

Synthesis of N-terminal end capped oligoglutamate: The method used is identical to that for synthesis of oligo(γ-Et-L-Glu) except for the following modifications. Although the stoichiometry of L-(Et)₂-Glu.HCl and end-capping substrate was varied, the total concentration of monomer and end-capping substrate remained at 2.5 mmol. Due to the insolubility of the ‘N’ acrylated monomers in aqueous buffer at pH 8 and 9, water miscible co-solvent (30% MeOH v/v) was added to obtain a clear solution, correspondingly the total monomer concentration and buffer concentration was changed to 0.125M and 0.25M respectively (based on results yet to be published) and solutions were titrated back to the desired pH using 10M NaOH or 10M HCl. Subsequently, 16 units/mL of the desired enzyme was added to catalyze oligopeptide synthesis. Precipitate formed was washed with 2×5 mL of deionized water then with an HCl solution (pH 2, 2×5 mL), separation of precipitate after each washing step was by centrifugation (5000 rpm), supernatants were discarded and the precipitate was freeze dried to obtain the product. This product was further vortexed with dichloromethane to dissolve any residual ‘N’ acrylated monomer. This product is further dried in vacuo. The %-yield of ‘N’ terminal end-capped chains of oligo(γ-L-Et-Glu) [oligo(γ-L-Et-Glu)-(E/F)—N-acryl] was calculated gravimetrically from the precipitated product obtained.

Synthesis of N-terminal modification of oligoglutamate post synthesis. One methodology envisaged involves activation of carboxyl groups of lauric acid, 0.36 g of lauric acid (1.8 mmol) and 0.2 g NHS (1.8 mmol) dissolved in 20 mL dimethyl sulfoxide (DMSO) in a dried 150 mL round bottom flask and treated with 0.47 g dicyclohexylcabodiimide (DCC, 2.3 mmol) and 0.28 g 4-dimethylaminopyridine (DMAP, 2.3 mmol) for 24 h at room temperature. The solid dicyclohexylurea formed was removed by filtration (this activation procedure can be performed with the list of fatty acid depicted in embodiment. For coupling of oligo(γ-L-Et-Glu), 1.8 mmol of activated lauric acid and 2.0 g oligo(γ-ethyl-1-glutamate, 1.5 mmol). In essence co-oligomers of L-(Et)2-Glu.HCl with other amino acid ester hydrochloride (which can be any of the amino acid depicted in chart 1) can be substituted in the coupling process to diversify the property of the product lipopeptide. After adding 0.6 mL of triethylamine (4.4 mmol), the reaction mixture was stirred at room temperature for 3 days. The product mixture was precipitated in excess cold water and centrifuged for about 30 min; the precipitate was then washed with 50 mL cold methanol to remove unreacted activated lauric acid. Dried lipopeptide powder (100 mg) was kept in 1 N NaOH solution (5 mL) at 60° C. for 36 h; the mixture was then neutralized with 6.0 M hydrochloride solution to afford amphiphilic lipopeptide containing desterified oligo(γ-L-Glu).

Supporting Information Available: Hydrolytic activity and protein content of proteases used; MALDI-TOF spectra of products formed using the following amine nucleophiles: 1-phenyl methanamine (BzA), 2-amino methylfuran (FMA), 5-methyl-2-amino methylfuran (MFMA), 4-methylamino pyridine (MPy), bicyclo[2.2.1]hept-5-en-2-ylmethanamine (NorbA), and L-phenylalaminol (FOH). This material is available free of charge via the Internet at http://pubs.acs.org.

The above detailed description of the preferred embodiments, examples, and the appended figures are for illustrative purposes only and are not intended to limit the scope and spirit of the invention, and its equivalents, as defined by the appended claims. One skilled in the art will recognize that many variations can be made to the invention disclosed in this specification without departing from the scope and spirit of the invention.

### REFERENCES

(1) Jurgen M. Smeenk, M. B. J. O., Jens Thies; David A. Tirrell, H. G. S., Jan C. M. van Hest Angew. Chem. Int. Ed. 2005, 44, 1968-1971.
(2) O'Brien-Simpson, N. M.; Ede, N. J.; Brown, L. E.; Swan, J.; Jackson, D. C. J. Am. Chem. Soc. 1997, 119, 1183-1188.
(3) Herrmann, A.; Mihov, G.; Vandermeulen, G. W. M.; Klok, H.-A.; Mullen, K. Tetrahedron 2003, 59, 3925-3935.
(4) Kim, S.; Healy, K. E. Biomacromolecules 2003, 4, 1214-1223.
(5) Tobias Heck; Hans-Peter E. Kohler; Michael Limbach; Oliver Flögel; Dieter Seebach; Birgit Geueke Chemistry and Biodiversity 2007, 4, 2016-2030.
(6) Orwig, K. S.; Dix, T. A. Tetrahedron Lett. 2005, 46, 7007-7009.
(7) Hadden, M. K.; Orwig, K. S.; Kokko, K. P.; Mazella, J.; Dix, T. A. Neuropharmacology 2005, 49, 1149-1159.
(8) Ambade, A. V.; Sandanaraj, B. S.; Klaikherd, A.; Thayumanavan, S. Polym. Int. 2007, 56, 474-481.
(9) Minkin, C.; Jennings, J. M. Science 1972, 176, 1031-1033.
(10) Ribeiro da Silva, M. A. V.; Santos, A. F. L. O. M. The Journal of Chemical Thermodynamics 2008, 40, 1451-1457.
(11) Proteinase catalyzed synthesis of peptide bonds Meister, A., Ed.; John Wiley & sons, New York, 1992; Vol. 53.
(12) Anne Ferjancic, A. P., Hubert Gaertner Appl. Microbiol. Biotechnol. 1990, 32, 651-657.
(13) Ferjancic, A.; Puigserver, A.; Gaertner, H. Biotechnol. Lett 1991, 13, 161-166.
(14) Uyama, H.; Fukuoka, T.; Komatsu, I.; Watanabe, T.; Kobayashi, S. Biomacromolecules 2002, 3, 318-323.
(15) Shuichi Matsumura; Yasuhiro Tsushima; Nobuyuki Otozawa; Saeko Murakami; Kazunobu Toshima; Graham Swift Macromol. Rapid Commun. 1999, 20, 7-11.
(16) Soeda, Y.; Toshima, K.; Matsumura, S. Biomacromolecules 2003, 4, 196-203.
(17) Li, G.; Raman, V. K.; Xie, W.; Gross, R. A. Macromolecules 2008, 41, 7003-7012.

1. A process for preparing oligopeptides end-functionalized at the N-terminus, C-terminus or at both ends that has the general formula oligomer of the formula CA-(AA)ₙ-B wherein B is a group at the carboxyl terminus, CA is a group at the N-terminus, AA is an amino acid and n is the oligomer chain length which comprises the steps of:
a) admixing one or more amino acid alkyl esters with one or more an end-functionalizing agents and one or more proteases in a reaction medium;
b) heating the mixture to between about 5° C. to about 90° C. for between 5 minutes and 24 hours; and
c) recovering the end-functionalized oligopeptide.

2. The process of claim 1 wherein the amino acid alkyl ester has the following general formula:
H₂N—CH(R)—(CR′H)ₙ—COOX (1)
in which
R represents an amino acid side chain,
R′ represents a different amino acid side chain present in α-amino acids,
X is an alkyl ester preferably consisting of an alkyl group selected from those containing from one to six carbon atoms but may consist of up to 20 carbon atoms, wherein (i) the alkyl ester may be straight or branched chain and include methyl, ethyl, propyl, isopropyl, butyl, hexyl and the like, (ii) preferably the alkyl ester is selected from the group consisting of methyl, ethyl or propyl groups, and (iii) activated esters can also be used in place of alkyl esters, (iv) examples of activated esters include guanadinophenyl, p-nitrophenyl, 1,1,1,3,3,3-hexafluoroisopropyl, 2,2,2-triifluoroethyl, 2-chloro ethyl ester, carbamoyl methyl ester, benzyl esters, and anilides.

3. The process of claim 2 wherein structure 1 has n=0, the stereochemistry is L, and R is selected from one or more of the natural amino acid side chains shown in Chart 1.

4. The process of claim 2 wherein structure 1 is selected from the family of non-natural amino acids and β-amino acids having a generalized structure of:
H₂NCH₂CH(R)—COOX (2)
wherein the β-amino acids and other non-natural amino acid structures useful as substrates for protease-catalyzed oligopeptide synthesis or protease-catalyzed coupling of preformed segments of oligo(amino acids).

5. The process of claim 4, wherein the β-amino acids are those that consist of the general structure (2) and R is selected from one or more of the natural amino acid side chains shown in Chart 1.

6. The process of claim 4, wherein the β-amino acids are selected from the group consisting of β-alanine, L-β-homotyrosine, L-β-homoleucine, L-β-homoisoleucine and L-β-homotryptophan.

7. The process of claim 4, wherein the non-natural amino acid esters are selected from the group consisting of carnitine [3-Hydroxy-4-trimethylammonio-butanoate], ornithine [(+)-(S)-2,5-diamino valeric acid], citruline [2-Amino-5-(carbamoylamino)pentanoic acid], 4-aminobutanoic acid and L-Dopamine, and other non-natural amino acids.

8. The process of claim 2 wherein the alkyl ester group X in structure (1) is ethyl.

9. The process of claim 1 wherein the reaction medium consists of a phosphate, acetate, borate, carbonate, HEPES, or sulphate buffers with concentrations that are between 0.1M to 1.5M.

10. The process of claim 1, wherein amines are used to maintain reaction medium pH.

11. The process of claim 9 wherein a water-miscible cosolvent selected from the group consisting of formamides, alcohols (1°, 2°, 3°), dimethyl sulfoxide, tetrahydrofuran, acetone, acetonitrile, 1,2-ethylene glycol, 1,3-propylene glycol, or 1,4-butanediol is added in concentrations from 0 to 50%-v/v.

12. The process of claim 1 wherein the enzyme or enzyme mixture is selected from a member of a hydrolytic enzyme family that is further comprised of proteases, lipases, esterases and cutinases.

13. The process of claim 12 wherein the enzyme is selected from members of the protease family, and wherein
(i) the protease is selected from the group consisting of papain, bromelain, α-chymotrypsin, trypsin, Multifect P-3000 (Genencor), Purafect prime L (Genencor), alkaline protease (Genencor), metalloprotease (thermolysin), protease from subtilisin (family), pronasel, glutaminase, carboxypeptidase Y, clostrapin, protease from aspergillus oryzae species, pepsin, cathepsin, ficin, alcalase, carboxypeptidase, calpains, actinidin, chymosin, carbonic anhydrase, nonribosomal peptide synthetase, thrombin, cardosins A or B or pronase,
(ii) the reaction is catalyzed by one or a mixture of at least two proteases, and
(iii) variants of these enzymes, generated by standard protein engineering methods such as error-prone PCR and gene shuffling are used to further improve a proteases activity and selectivity for use in the current invention.

14. The process of claim 12 wherein enzymes are added to the reaction media as enzyme powders, in solution, or immobilized on a support.

15. The process of claim 1 wherein the reaction is terminated by filtration of the immobilized enzyme.

16. The process of claim 1 wherein the reaction is terminated by separation of the precipitated end-functionalized oligopeptide product by filtration or centrifugation from the enzyme remaining in the reaction medium.

17. The process of claim 1 wherein the reaction is terminated by using a membrane with a suitable pore size that separates a soluble end-functionalized oligopeptide product from the soluble enzyme.

18. The process of claim 1 wherein the reaction is terminated by selective precipitation of either the soluble enzyme or the soluble end-functionalized oligopeptide product.

19. The process of claim 1 wherein the reaction time is between 5 minutes and 24 hours.

20. The process of claim 1 wherein the reaction time is between 10 minutes and 8 hours.

21. The process of claim 1 wherein the reaction time is between 30 minutes and 3 hours.

22. The process of claim 1 wherein the reaction temperature is between 5° C. and 90° C.

23. The process of claim 1 wherein the reaction temperature is between 25° C. and 60° C.

24. The process of claim 1 wherein the reaction temperature is between 30° C. and 40° C.

25. The process of claim 1 wherein the reaction is performed by passing reactants through a column wherein the stationary phase consists of the immobilized enzyme.

26. The process of claim 1 wherein the end-capped oligopeptides consist of a mixture of oligomers where the average chain length, determined by measuring the number average molecular weight, ranges from 2 to 100 units.

27. The process of claim 1 wherein the end-capped oligopeptides consist of a mixture of oligomers where the average chain length, determined by measuring the number average molecular weight, ranges from 5 to 50 units.

28. The process of claim 1 wherein the end-capped oligopeptides consist of a mixture of oligomers where the average chain length, determined by measuring the number average molecular weight, ranges from 10 to 20 units.

29. The process of claim 1 wherein the end-capped oligopeptides consist of a mixture of oligomers with a polydispersity, determined by dividing the weight average molecular weight by the number average molecular weight, of 50.

30. The process of claim 1 wherein the end-capped oligopeptides consist of a mixture of oligomers with a polydispersity, determined by dividing the weight average molecular weight by the number average molecular weight, that is <25.

31. The process of claim 1 wherein the end-capped oligopeptides consist of a mixture of oligomers with a polydispersity, determined by dividing the weight average molecular weight by the number average molecular weight, that is <5.

32. The process of claim 1 wherein the end-capped oligopeptides consist of a mixture of oligomers with a polydispersity, determined by dividing the weight average molecular weight by the number average molecular weight, that is <1.5.

33. The process of claim 1 wherein the end-functionalizing agent consists of a member selected from the family of primary amines.

34. The process of claim 33 wherein the end-functionalizing agent is selected from a primary amine having the structure R—(CH₂)ₙ—NH₂, where R belongs to a member of the family of 5 or 6 membered rings.

35. The process of claim 34 wherein R is an aromatic 5 or 6 membered ring.

36. The process of claim 35 wherein R is an aromatic heterocyclic 5 or 6 membered ring that contains a sulfur, oxygen or nitrogen atom.

37. The process of claim 35 wherein R is a phenyl ring with one or more substituents that can be selected from the group consisting of: —N₃, —NO₂, —OH, —F, —Cl, —I, —COOH, —CH₂═CH, —CH≡C—H.

38. The process of claim 34 wherein n is either 1 or 2.

39. The process of claim 34 wherein n is 1.

40. The process of claim 1 wherein the end-functionalization agent consists of an activated ester with the general formula:
Y′Y—[H]ₐN—CH(R)—(CR′H)ₙ—COOX (3)
wherein
X is an alkyl ester preferably consisting of an alkyl group selected from those containing from one to six carbon atoms but may consist of up to 20 carbon atoms, wherein (i) the alkyl ester may be straight or branched chain and include methyl, ethyl, propyl, isopropyl, butyl, hexyl and the like, (ii) preferably the alkyl ester is selected from the group consisting of methyl, ethyl or propyl groups, and (iii) activated esters can also be used in place of alkyl esters, (iv) examples of activated esters include guanadinophenyl, p-nitrophenyl, 1,1,1,3,3,3-hexafluoroisopropyl, 2,2,2-triifluoroethyl, 2-chloro ethyl ester, carbamoyl methyl ester, benzyl esters, and anilides, Y and/or Y′ is selected from H, methyl, ethyl, CH₂═CH—CO—, CH₂═C(CH₃)—CO—, HOOC—CH═CH—CO— (cis or trans) and other polymerizable groups wherein polymerizable groups require a polymerization method selected from the group consisting of conventional free radical polymerization, ATRP, RAFT, and ring-opening metathesis reactions, functional groups used for photolytic crosslinking, a cinnamoyl (Ph-CH═CH—CO—) group, structures that are crosslinkable via redox catalysts that are of chemical or enzyme (e.g. laccases, peroxidases) origin, and HO-Ph-(CH₂)—CO— where the hydroxyl group is at the para-position.

41. The process of claim 40, wherein n=0, R is selected from a member of the natural 20 amino acid side chains shown in Chart 1, Y is H, and Y′ is CH₂═CH—CO— or CH₂═C(CH₃)—CO—.

42. A process for preparing oligopeptides end-functionalized at the N-terminus, C-terminus or at both ends that has the general formula C-(AA)ₙ-B wherein B is a group at the carboxyl terminus, C is a group at the N-terminus, AA is an amino acid and n is the oligomer chain length which comprises the steps of:
a) admixing one or more amino acid alkyl esters one or more proteases in a reaction medium;
b) heating the mixture to between about 5° C. to about 90° C. for between 5 minutes and 24 hours;
c) recovering the oligopeptide; and
d) performing a modification of the N-terminal amino group by conventional coupling methods using conventional chemical methods well known by persons of ordinary skill in the art.

43. The process of claim 42 wherein the N-terminal group of oligopeptides is modified by N-acylation chemistry.

44. The process of claim 43 where the N-acylated amino acids formed have the following general structure:
R(C═O)NH[AA]ₙCOOX (4)
wherein R(C═O) can be derived from any of the following natural fatty acids:

| | Carbon | Double | | Common |
|---|---|---|---|---|
| Common Name | Atoms | Bonds | Scientific Name | Sources |
| lauric acid | 12 | 0 | dodecanoic acid | coconut oil |
| (LA) | | | | |
| myristic acid | 14 | 0 | tetradecanoic acid | palm kernel |
| (MA) | | | | oil |
| palmitic acid | 16 | 0 | hexadecanoic acid | palm oil |
| (PA) | | | | |
| palmitoleic | 16 | 1 | 9-hexadecenoic acid | animal fats |
| acid (POA) | | | | |
| stearic acid (SA) | 18 | 0 | octadecanoic acid | animal fats |
| oleic acid (OA) | 18 | 1 | 9-octadecenoic acid | olive oil |
| ricinoleic acid | 18 | 1 | 12-hydroxy-9- | castor oil |
| (RA) | | | octadecenoic acid | |
| linoleic acid (LA) | 18 | 2 | 9,12-octadeca- | grape seed |
| | | | dienoic acid | oil |
| α-linolenic | 18 | 3 | 9,12,15-octadeca- | flaxseed oil |
| acid (ALA) | | | trienoic acid | (linseed oil) |
| γ-linolenic | 18 | 3 | 6,9,12-octadeca- | borage oil |
| acid (GLA) | | | trienoic acid | |
| behenic acid (BA) | 22 | 0 | docosanoic acid | rapeseed oil |
| erucic acid (EA) | 22 | 1 | 13-docosenoic acid | rapeseed oil |

45. The process of claim 44 where the fatty acid is first modified by hydrogenation, epoxidation, hydroxylation, prior to reaction with NH₂ terminal groups of oligopeptides.

46. The process of claim 44 wherein R is selected from the group consisting of —CH₂═CH—CO—, CH₂═C(CH₃)—CO—, HOOC—CH═CH—CO— (cis or trans).

47. The process of claim 42 wherein the amino acids consists primarily of glutamic acid units (>50 mol % of repeat units) and the other 50 mol % of repeat units is selected from one or a mixture of two or more naturally occurring amino acids.
